(19) United States
(12) Patent Application Publication
(10) Pub. No.: US 2024/0216106 A1
(43) Pub. Date: Jul. 4, 2024

(54) METHODS AND APPARATUSES INCLUDING TOOTH ERUPTION PREDICTION

(71) Applicant: Align Technology, Inc., San Jose, CA (US)

(72) Inventors: Jeeyoung CHOI, Sunnyvale, CA (US); Palak MITTAL, Newark, CA (US); Yuxiang WANG, Newark, CA (US); Xiaogang WANG, Wake Forest, NC (US); Zhaonan SUN, Charlottesville, VA (US); Yi-Lin CHIU, Foster City, CA (US); Kangning SU, Pullman, WA (US); Bastien PESENTI, San Jose, CA (US); Christopher E. CRAMER, Durham, NC (US); Ravi PATEL, San Jose, CA (US); Ken WU, San Jose, CA (US); Konstantin STOLYAROV, Novosibirsk (RU); Vladimir CHISTYAKOV, Moscow (RU)

(21) Appl. No.: 18/404,867

(22) Filed: Jan. 4, 2024

Related U.S. Application Data
(60) Provisional application No. 63/478,495, filed on Jan. 4, 2023.
(60) Provisional application No. 63/581,278, filed on Sep. 7, 2023.

(51) Int. Cl. A61C 7/00; A61C 7/08; G06T 7/00; G16H 10/60

(52) U.S. Cl. CPC A61C 7/002; A61C 7/08; G06T 7/00; G16H 10/60; G06T 2207/10116; G06T 2207/20081; G06T 2207/20084; G06T 2207/30036

(57) ABSTRACT

Apparatuses and methods for assessing a dental x-ray image and predicting tooth eruption and/or exfoliation. The apparatuses may use one or more trained neural networks to assess a patient's x-rays as well as dental measurements determined from the patient's x-rays to predict approximately when a permanent tooth may erupt to replace a baby tooth. The neural networks may be trained using a data training set of x-ray images and accompanying dental measurement data.

### CLAIM OF PRIORITY

This patent application claims priority to U.S. Provisional Patent Application No. 63/478,495, titled “TOOTH ERUPTION PREDICTION,” filed on Jan. 4, 2023, and U.S. Provisional Patent Application No. 63/581,278, titled “METHODS AND APPARATUSES INCLUDING TOOTH ERUPTION PREDICTION,” filed on Sep. 7, 2023, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates generally to dental case assessment, and more specifically to predicting tooth eruption and/or exfoliation in a patient.

### BACKGROUND

Orthodontic and dental treatments using a series of patient-removable appliances (e.g., “aligners”) are very useful for treating patients. Treatment planning is typically performed in conjunction with the dental professional (e.g., dentist, orthodontist, dental technician, etc.), by generating a model of the patient's teeth in a final configuration and then dividing the treatment plan into a number of intermediate stages (steps) corresponding to individual appliances that are worn sequentially. This process may be interactive, adjusting the staging and in some cases the final target position, based on constraints on the movement of the teeth and the dental professional's preferences. Once the final treatment plan is finalized, the series of aligners may be manufactured corresponding to the treatment planning.

Treatment planning for young patients (e.g., patients between the ages of 5-14 years) may be complicated by the eruption of permanent teeth that replace the patient's baby teeth. Tooth eruption may cause spaces to form within a dental arch that affect tooth placement and movement. In some cases, an erupting tooth may cause a treatment plan to be revised, costing money, and perhaps extending treatment time. If the timing of a tooth eruption can be predicted, then a clinician (Dentist, Orthodontist, or the like) can ensure that the treatment plan can accommodate the erupting teeth. In addition, predicting tooth eruption and exfoliation may be difficult, especially because of the high variability of tooth eruption times across a variety of different patient demographics. In some cases, the only diagnostic data available may be a patient's x-ray images. Predicting tooth eruption times based on a patient's x-ray images may be difficult due to limited two-dimensional information.

Thus, there is a need for predicting tooth eruptions and/or exfoliations based on dental x-ray images.

### SUMMARY OF THE DISCLOSURE

Described herein are apparatuses (e.g., systems and devices) and methods that can assess a patient's x-ray image, and predict a patient's tooth eruption and/or a patient's tooth exfoliation. In some examples, these apparatuses and methods may include a machine learning agent (e.g., neural network) that is trained to predict tooth eruption and/or tooth exfoliation.

In general, described herein are methods of assessing a patient's x-ray image. Any of these methods may include receiving one or more patient x-ray images, determining one or more dental measurements associated with the one or more patient x-ray images and, using a trained neural network, predicting tooth eruption and/or tooth exfoliation. Any of these methods may include forming one or more (e.g., a series) of dental appliances that are configured to fit over the patient's dentition and that include one or more accommodations for the eruption/exfoliation.

In general, a patient's x-ray image may include a two-dimensional x-ray such as a panoramic x-ray or one or more bitewing x-rays. In some cases, a patient's x-ray image may include a three-dimensional x-ray such as conventional computer tomography or cone-beam computed tomography. In some variations, a patient's x-ray images may include both two-dimensional and three-dimensional images.

In general, tooth eruption and tooth exfoliation may be predicted by the trained neural network. In general, the trained neural network may be trained with training data. The training data may include one or more x-ray images as well as dental measurements that are associated with each of the x-ray images. The dental measurements may describe various attributes, measurements, characteristics, and the like of the patient's dentition. For example, the dental measurements may include a tooth's crown to gingival distance, a relative distance between an unerupted tooth and a medial tooth's crown, or other measurements.

In general, a dental treatment plan may be formulated or modified based on a predicted tooth eruption or tooth exfoliation. In some cases, a dental treatment plan may include a series of dental aligners. One or more of the dental aligners may be modified to accommodate an erupting permanent tooth.

Described herein are methods for predicting a change in a patient's dentition. The methods may include receiving dental measurements determined from one or more x-ray images of a patient, predicting a change in a patient's dentition based on the dental measurements using a trained neural network, wherein the trained neural network is trained using a plurality of training x-ray images and corresponding patient data, and generating a treatment plan based on the predicted change in the patient's dentition. Notably, a change in the patient's dentition may include a tooth eruption, a tooth exfoliation, or a combination thereof.

In general, the dental measurements may be associated with the measurements of any feasible characteristics of a patient's dentition. In some cases, the measurements may include a measurement from a crown of a tooth to a gingival feature. In some other cases, the measurements may include a relative distance between an unerupted tooth and a medial tooth's crown.

In any of the methods described herein, the corresponding patient data may include patient age, patient gender, patient weight, patient ethnicity, patient geographic location, or a combination thereof. The patient data may be used to train the neural networks and, in some instances, may be used during execution of a neural network to predict tooth eruption and/or exfoliation.

The corresponding patient data may also include facial measurements. Facial measurements may include the distances between any two or more facial datums or landmarks that may be identified on a patient's face.

In general, a treatment plan includes a plurality of dental aligners that are configured to accommodate an erupting tooth. In some variations, the dental aligners may include a cutout region near a predicted location of an erupting tooth. In some other variations, the dental aligners may include an enlarged pocket disposed in a region near the predicted erupting tooth. In still other variations, the dental aligners may include an undercut region to accept an erupting tooth.

In any of the methods described herein, the x-ray images of the patient may include bitewing x-rays, panoramic x-rays, or a combination thereof.

In general, any of the methods described herein may also include receiving updated dental measurements determined from one or more subsequent x-ray images of the patient and updating the prediction of the change in a patient's dentition based at least in part on the updated dental measurements. In some variations, the updated dental measurement may include measurements determined from an intraoral scan.

Also described herein are apparatuses configured to perform any of these methods. For example, described herein is an apparatus for predicting a change to a patient's dentition that may include a communication interface, one or more processors, and a memory storing instructions that, when executed by the one or more processors, causes the one or more processors to receive dental measurements determined from one or more x-ray images of a patient, predict a change in a patient's dentition based on the dental measurements using a trained neural network, wherein the trained neural network is trained using a plurality of training x-ray images and corresponding patient data, and generate a treatment plan based on the predicted change in the patient's dentition.

Also described herein is a non-transitory computer-readable storage medium storing instructions that, when executed by one or more processors of a device, cause the device to receive dental measurements determined from one or more x-ray images of a patient, predict a change in a patient's dentition based on the dental measurements using a trained neural network, wherein the trained neural network is trained using a plurality of training x-ray images and corresponding patient data, and generate a treatment plan based on the predicted change in the patient's dentition.

Also described herein is a method for predicting a change in a patient's dentition that includes receiving a reference 2D x-ray image and a corresponding reference 3D x-ray image, wherein the reference 2D x-ray image and the reference 3D x-ray image are based on an ideal arch describing ideal tooth positions, determining a transformation function to map the 2D x-ray image to the 3D x-ray image, receiving a patient's 2D x-ray image, generating a predicted 2D model based on mapping the patients 2D x-ray image to the reference 2D x-ray image, generating a predicted 3D model based on the transformation function, and predicting a change in a patient's dentition based on the predicted 3D model.

In general, the method may also include generating a treatment plan based on the predicted change in the patient's dentition. Also in general, the change in the patient's dentition may include a tooth eruption, a tooth exfoliation, or a combination thereof.

In some variations, any of the methods may include predicting a geometry of an unerupted tooth based on statistical modelling. In general, the patient's 2D x-ray image may be a panoramic x-ray, a series of bitewing x-rays, a series of periapical x-rays, or a combination thereof.

In general, in any of the methods described herein, the ideal arch may be selected based on a patient's demographics.

In general, in any of the methods described herein the 3D x-ray image may be a computer tomography (CT) or a cone-beam computed tomography (CBCT) scan.

Also described herein is an apparatus for predicting a change to a patient's dentition that may include a communication interface, one or more processors, and a memory storing instructions that, when executed by the one or more processors, causes the one or more processors to receive a reference 2D x-ray image and a corresponding reference 3D x-ray image, wherein the reference 2D x-ray image and the reference 3D x-ray image are based on an ideal arch describing ideal tooth positions, determine a transformation function to map the 2D x-ray image to the 3D x-ray image, receive a patient's 2D x-ray image, generate a predicted 2D model based on mapping the patients 2D x-ray image to the reference 2D x-ray image, generate a predicted 3D model based on the transformation function, and predict a change in a patient's dentition based on the predicted 3D model.

Also described herein is a non-transitory computer-readable storage medium storing instructions that, when executed by one or more processors of a device, cause the device to receive a reference 2D x-ray image and a corresponding reference 3D x-ray image, wherein the reference 2D x-ray image and the reference 3D x-ray image are based on an ideal arch describing ideal tooth positions, determine a transformation function to map the 2D x-ray image to the 3D x-ray image, receive a patient's 2D x-ray image, generate a predicted 2D model based on mapping the patients 2D x-ray image to the reference 2D x-ray image, generate a predicted 3D model based on the transformation function, and predict a change in a patient's dentition based on the predicted 3D model.

For example, described herein are methods comprising: extracting or receiving dental measurements from one or more x-ray images of a patient; predicting a tooth eruption and/or exfoliation in the patient's dentition based on the dental measurements using a trained neural network, wherein the trained neural network is trained using a plurality of training x-ray images and corresponding patient data to generate a probability value indicating the likelihood of eruption and/or exfoliation of a tooth at a particular location; and forming a dental appliance configured to fit over the patient's dentition and including an accommodation to accommodate the predicted tooth eruption and/or exfoliation, wherein the accommodation comprises one or more of: a cutout region configured to accommodate the predicted tooth eruption and/or exfoliation, a pocket configured to accommodate the predicted tooth eruption and/or exfoliation, and/or an undercut region configured to accommodate the predicted tooth eruption and/or exfoliation.

Any of these methods may include generating a treatment plan based on the probability value.

As mentioned, the dental measurements may include a crown to gingival distance for at least one tooth. In some examples the dental measurements include a relative distance between an unerupted tooth and a medial tooth's crown. The corresponding patient data may include patient age, patient gender, patient weight, patient ethnicity, patient geographic location, or a combination thereof. The corresponding patient data may include facial measurements associated with two or more facial datums. The x-ray images of the patient may include panoramic x-rays, bitewing x-rays, or a combination thereof.

Any of these methods may include receiving updated dental measurements determined from one or more subsequent x-ray images of the patient; and updating the prediction of the change in a patient's dentition based at least in part on the updated dental measurements. The updated dental measurements may include measurements determined from an intraoral scan.

Any of these methods may include outputting the probability value for the particular location.

In general, forming the dental appliance may include forming a digital model of the dental appliance (or a series of appliances) for the upper and/or lower teeth. In some examples, forming the dental appliance may include fabricating the dental appliance. For example, fabricating the dental appliance may include fabricating the dental appliance by a direct fabrication process (e.g., 3D printing, etc.).

In any of these methods and apparatuses, the dental appliance may comprise a patient-removable aligner configured to fit over the patient's dentition.

Forming the dental appliance may include generating a digital model of the dental appliance and positioning the accommodation on the dental appliance in a region configured to be worn over the particular location. The dental appliance may include one or more tooth-accommodating cavities configured to be worn over the patient's dentition (e.g., teeth).

Also described herein are apparatuses for performing any of these methods. For example, and apparatus may include: one or more processors; and a memory storing instructions that, when executed by the one or more processors, causes the apparatus to perform the method comprising: extracting or receiving dental measurements from one or more x-ray images of a patient; predicting a tooth eruption and/or exfoliation in the patient's dentition based on the dental measurements using a trained neural network, wherein the trained neural network is trained using a plurality of training x-ray images and corresponding patient data to generate a probability value indicating the likelihood of eruption and/or exfoliation of a tooth at a particular location; forming a dental appliance configured to accommodate the predicted tooth eruption and/or exfoliation, wherein the dental appliance comprises one or more of: a cutout region configured to accommodate the predicted tooth eruption and/or exfoliation, a pocket configured to accommodate the predicted tooth eruption and/or exfoliation, and/or an undercut region configured to accommodate the predicted tooth eruption and/or exfoliation.

In some examples the method may include: positioning one or more jaw arch splines relative to a patient's dental arch in a virtual model of the patient's dental arch; positioning one or more buccal-lingual planes in a region of the patient's dental arch to receive erupting teeth; determining, for each buccal-lingual plane, a two-dimensional (2D) tooth profile based on a projection of the one or more jaw arch splines through the one or more buccal-lingual planes; and forming a dental aligner configured to be worn over the patient's dental arch, wherein forming includes setting a void region for the dental aligner based on the 2D tooth profiles from the one or more buccal-lingual planes.

The one or more jaw arch splines may pass through an exterior point on a surface of existing teeth of the patient. The one or more jaw arch splines may be associated with at least one of a buccal gingiva point or a lingual gingiva point on a surface of an existing tooth of the patient. The one or more jaw arch splines may be associated with at least one of a buccal cusp, a lingual cusp, or a groove arch points on a surface of an existing tooth of the patient. The one or more jaw arch splines may be described by piecewise continuous polynomial that describes a curve passing through a predetermined point of an existing tooth of the patient. In some examples the one or more buccal-lingual planes are normal to a patient's dental arch. Determining the void may include forming a volume based on the projection of the one or more jaw arch splines through the one or more buccal-lingual planes. In some examples determining the one or more jaw arch splines comprises adjusting, by a clinician, the one or more jaw arch splines.

Adjusting may be performed by the clinician interacting with a graphical user interface. Any of these methods may include displaying the one or more jaw arch splines on a graphical user interface. Any of these methods may include displaying the dental aligner including the void on a display.

In general, any of these methods may include modifying the dental aligner to accommodate teeth from a different dental arch.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a graph that illustrates an example probability of tooth exfoliation versus age for young patients.

FIG. 2 schematically illustrates one example of a machine-learning tooth eruption prediction apparatus.

FIG. 3 is a flowchart showing an example method for training a neural network to predict tooth eruption.

FIG. 4 is a flowchart showing one example of a method for predicting tooth eruption for a patient using the machine learning approach of FIG. 3.

FIG. 5 is a flowchart showing one example of a method for predicting tooth eruption location and direction for a patient.

FIG. 6 is a flowchart showing an example method for training a neural network to predict tooth eruption based on facial features.

FIG. 7 shows an illustration of a face with the location of possible facial datums.

FIG. 8 is a flowchart showing one example of a method for predicting tooth eruption for a patient using the machine learning approach of FIG. 6.

FIGS. 9A-9D show example dental aligners that are designed to accommodate an erupting tooth.

FIG. 10 shows a block diagram of a device that may be one example of the machine-learning tooth eruption prediction apparatus of FIG. 2.

FIG. 11A is an x-ray scan image illustrating an example of a predicting as described herein.

FIG. 11B illustrates one example of a method of predicting based on rescan timing as described herein.

FIG. 12 schematically illustrates an example of a workflow to predict the location and direction of erupting permanent teeth using X-ray images as described herein.

FIG. 13 shows a simplified dental arch illustrating another approach to accommodate unerupted teeth for aligner designs.

FIG. 14 shows an example buccal-lingual plane.

FIGS. 15A and 15B illustrate an example usage of buccal-lingual planes and jaw arch splines.

FIG. 16 shows a partial dental arch that may be displayed on a graphical user interface.

FIG. 17 is a flowchart showing one example of a method for determining a void in a dental aligner.

FIG. 18 is a diagram illustrating an example of a computing environment including the tooth eruption prediction methods and apparatuses, e.g., module(s), described herein.

### DETAILED DESCRIPTION

Images, such as dental images, are widely used in the formation and monitoring of a dental treatment plan. For example, some dental images may be used to determine a starting point of a dental treatment plan, or in some cases determine whether a patient is a viable candidate for any of a number of different dental treatment plans. The output of the apparatuses and methods described herein may be based on the age of the patient and the complexity of their treatment goals, and may provide the clinician (doctor, dentist, orthodontist, etc.) valuable insight to decide whether or not to extract or otherwise modify the treatment.

Described herein are apparatuses (e.g., systems and devices, including software) and methods for training and applying a machine learning agent (e.g., a neural network) to assess a patient's dental images, including x-ray images, to predict a patient's tooth eruption and/or exfoliation. In particular, these methods and apparatuses may predict the timing of eruption, the location of the eruption, the direction that the tooth may erupt and in some cases the geometry of the erupting tooth. These methods and apparatuses may be particularly helpful when used in combination with or as part of dental treatment plan, which may be divided up into steps (e.g., stages, segments, keyframes, etc.). The resulting predictions may be made in the context of the stages of the proposed or ongoing treatment plan. Thus, these methods and apparatuses may use data from the patient (e.g., imaging data) to predict when an eruption of a tooth (or teeth) will happen, including in some examples, which stage or segment, and may further predict how long the tooth will take to erupt, where it will erupt, etc.

In any of these examples the dental image may be a patient's dental x-ray image which may include one or more of a panoramic, a bitewing, a periapical, or any other feasible x-ray image. In addition, measurement data, determined from the patient's dental x-ray may be included. Example measurement data may include a tooth's crown to gingival distance, a relative distance between an unerupted tooth and a medial tooth's crown, and the like. A processor or processing node can execute one or more neural networks that have been trained to predict tooth eruption and/or exfoliation from the patient's dental x-rays.

Also described herein are apparatuses and methods for estimating tooth eruption and exfoliation using a reference two-dimensional dental arch and a reference three-dimensional dental arch. A patient's two-dimensional x-ray is mapped to the reference three-dimensional arch to predict motion of the patient's teeth.

After determining an estimated time of tooth eruption or exfoliation, a clinician may change one or more aspects of a dental treatment plan. For example, based on a predicted eruption time, a clinician may decide to extract a baby tooth. In another example, one or dental aligners may be modified or adjusted to accommodate an erupting tooth.

Also described herein are methods, the method comprising: extracting or receiving dental measurements from one or more x-ray images of a patient; predicting a tooth eruption in the patient's dentition based on the dental measurements using a trained neural network, wherein the trained neural network is trained using a plurality of training x-ray images and corresponding patient data to generate a predicted shape of an erupting tooth at a particular location; and outputting the predicted shape of the erupting tooth for the particular location. These methods may be methods of determining the shape (e.g., morphology) of an erupting/unerupted tooth using the techniques described herein.

These methods may include any of the steps described herein. For example, any of the methods described herein may include (optionally) generating a treatment plan based on the predicted shape. In any of these methods, the method may include fabricating (e.g., by a direct fabrication process, etc.) one or more dental appliance, e.g., aligner, from any of the treatment plans or otherwise.

Also described herein are apparatuses and methods for determining the presence, size and/or position of a space (e.g., a void) in a dental aligner for receiving erupting teeth.

For example, described herein are methods comprising: positioning one or more jaw arch splines relative to a patient's dental arch in a virtual model of the patient's dental arch; positioning one or more buccal-lingual planes in a region of the patient's dental arch to receive erupting teeth; determining, for each buccal-lingual plane, a two-dimensional (2D) tooth profile based on a projection of the one or more jaw arch splines through the one or more buccal-lingual planes; and setting a void region of a dental aligner based on the 2D tooth profiles from the one or more buccal-lingual planes.

One or more jaw arch splines may be defined based on characteristics of the patient's existing teeth. These jaw arch splines may be used to determine a profile of the void. In some examples, one or more jaw arch splines may be projected onto one or more buccal-lingual planes that are positioned (virtually) in a region of a patient's dental arch that has or will have erupting teeth. The projections may be used to determine a curve on the buccal-lingual plane. Through the use of several curves, the profile or shape of the void may be determined.

As described herein, a method for determining a void in a dental aligner to receive erupting teeth may include determining one or more jaw arch splines for a patient's dental arch, determining positions for one or more buccal-lingual planes, wherein the buccal-lingual planes are disposed in a region of the patient's dental arch to receive erupting teeth, determining, for each buccal-lingual plane, a two-dimensional (2D) tooth profile based on a projection of the one or more jaw arch splines through the one or more buccal-lingual planes, and determining a void for a dental aligner based on the 2D tooth profiles from the one or more buccal-lingual planes.

In any of the methods described herein, the one or more jaw arch splines can pass through an exterior point on a surface of existing teeth of the patient. In general the exterior point may be selected from any feasible point on the existing tooth. The exterior points may be associated with external tooth features that affect the tooth's external profile or shape. Thus, the jaw arch splines may be associated with any external tooth features.

For example, in any of the methods described herein the one or more jaw arch splines may be associated with at least one or a buccal gingiva point or a lingual gingiva point on a surface of an existing tooth of the patient. In some other examples, in any of the methods described herein the one or more jay arch splines may be associated with at least one of a buccal cusp, a lingual cusp, or a groove arch points on a surface of the patient's existing teeth

In general, a jaw arch spline may be a mathematical equation that describes a curve that passes through selected exterior points on the patient's teeth. Often, the jaw arch spline may be a piecewise continuous polynomial that describes a curve passing through a predetermined point of an existing tooth of the patient.

As described herein, the buccal-lingual planes may be disposed in a region that has, or will have, erupting teeth. In general, the buccal-lingual planes may be normal to a dental arch.

In general, the volume or shape of the void may be based on the projection of the one or more jaw arch splines through the one or more buccal-lingual planes. For example, a curve on a buccal-lingual plane may be based one where one or more jaw arch splines intersect the buccal-lingual plane. Many curves on many buccal-lingual planes may be used to determine a profile of the void.

In any of the methods described herein, one or more of the jaw arch splines may be adjusted by a clinician. Through the adjustment of a jaw arch spline, the user can change or modify a shape of the void. In some examples, a graphical user interface may display one or more jaw arch splines as well as control points that allow a clinician to interact and change any jaw arch spline. After changing or modifying a jaw arch spline, the changes to the void may be displayed on the graphical user interface.

In any of the methods described herein, a dental aligner that contains a void can be modified to accommodate teeth from a different dental arch. For example, space that may be occupied by opposing teeth may be removed from the void to reduce or eliminate collisions between the dental aligner and the opposing teeth.

Described herein are apparatuses (e.g., systems and devices, including software) for determining a void in a dental aligner. The apparatuses may include one or more processors and a memory storing instructions that, when executed by the one or more processors, causes the apparatus to determine one or more jaw arch splines for a patient's dental arch, determine positions for one or more buccal-lingual planes, wherein the buccal-lingual planes are disposed in a region of the patient's dental arch to receive erupting teeth, determine, for each buccal-lingual plane, a two-dimensional (2D) tooth profile based on a projection of the one or more jaw arch splines through the one or more buccal-lingual planes, and determine a void for a dental aligner based on the 2D tooth profiles from the one or more buccal-lingual planes.

Also described herein are a non-transitory computer-readable storage medium storing instructions that, when executed by one or more processors of a device, cause the device to determine one or more jaw arch splines for a patient's dental arch, determine positions for one or more buccal-lingual planes, wherein the buccal-lingual planes are disposed in a region of the patient's dental arch to receive erupting teeth, determine, for each buccal-lingual plane, a two-dimensional (2D) tooth profile based on a projection of the one or more jaw arch splines through the one or more buccal-lingual planes, and determine a void for a dental aligner based on the 2D tooth profiles from the one or more buccal-lingual planes.

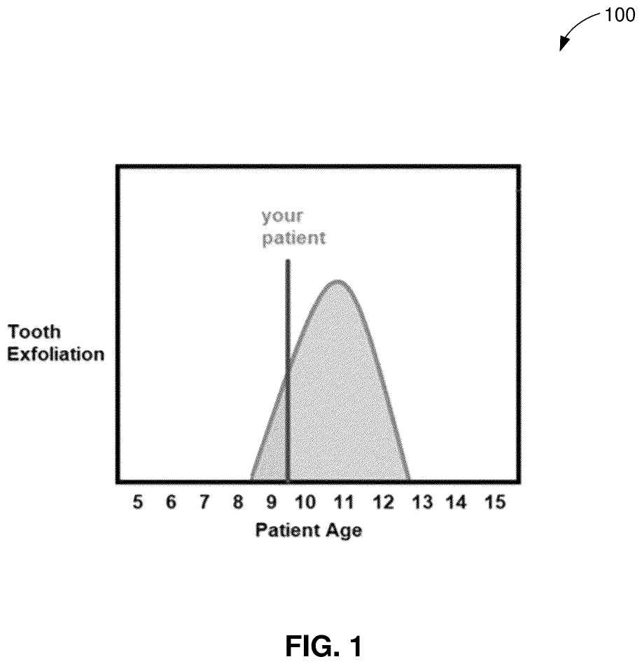

FIG. 1 is a graph 100 that illustrates an example probability of tooth exfoliation versus age for young patients. Tooth eruption and exfoliation may present complications in the formulation of treatment plans for younger patients. In some examples, a treatment plan may need modification depending on when tooth eruption and/or exfoliation occurs. In some cases, a clinician may desire to extract a baby tooth as part of a treatment plan to help ensure a good treatment outcome. In some other cases, dental aligners may need to be modified based on when eruption and/or exfoliation occurs.

As shown in the graph 100, tooth eruption happens most frequently around the age of 11, however the exact age may vary from patient to patient. Thus, a means for predicting when tooth eruption and/or exfoliation occurs can help a clinician formulate an efficient and effective treatment plan.

In general, when training a neural network to determine exfoliation as described herein, the network may be trained on juvenile (e.g., children less than 18 years old, between 6 and 18 years old, between 6-16 years old, etc.) data using a patient dataset in which the patient's within the training dataset were all undergoing treatment with dental aligners. Surprisingly, the use of such treatment-matched patient data is particularly helpful for these patients as it appears that the teeth of patients being treatment with dental aligners may exfoliate and erupt on a different schedule as compared to textbook data. In some cases, teeth may erupt sooner than expected based on textbook data, for example, when using a shell aligner system as compared to treatment with metal wires and bracket systems. This may be one reason for aligner misfitting complaints.

In general, the training database for the machine learning models described herein may include before and after patient scans, cross referenced by patient age; a predictability distribution table can be generated for each type of tooth from this data.

Although the methods and apparatuses described herein may be particularly helpful for children, these methods and apparatuses may be used for adult patients as well. For example, the prediction models included as part of the apparatuses and methods described herein may estimate a probability for an adult tooth to begin erupting as a replacement, and if that adult tooth would be fully erupted by the end of treatment. This would also be useful in how and when an eruption compensation feature is applied and designed, or design dynamically to match the eruption rate. For patients with partially erupted dentition, the methods and apparatuses may include how much further a tooth might erupt on its own, in remaining distance or as a completion percentage. As needed, clinicians may intervene by adding an attachment to further extrude the tooth or leave it alone to mitigate our known problem of excessive attachments use.

The methods and apparatuses described herein may be used for examining the existing patient dentition and generating a patient-specific growth pattern time-table for all of the remaining development activities still remaining. This may also include predicting remaining mandibular jaw growth development if the proper anatomical landmarks are detectable and these apparatus and methods may cross reference these tooth position against available submitted X-rays.

The methods and apparatuses (including software) described herein may generally be configured to predict eruption of one or more teeth. These methods and apparatuses may be methods for indicating the likelihood of success of a treatment based on the eruption prediction. In some cases the methods and apparatuses may provide as output (or partial output) a recommendation to proceed or not to proceed. For example, the method and apparatuses described herein may estimate a probability of exfoliation occurring after the treatment is over; in some examples the method or apparatus may provide a recommendation based on the probability of exfoliation occurring after the treatment is over; e.g., if this probably is greater than a threshold (post-treatment eruption threshold), e.g., X %, then the recommendation is not to perform the treatment or to discontinue treatment (if it has begun). Any of these methods and apparatuses may determine a probability of exfoliation occurring within the next N months. In some examples, if the probably of exfoliation occurring within the next N months is greater than a threshold (predicted exfoliation threshold), e.g., Y %, then a recommendation is to extract the intervening tooth. Any of these methods may determine a probability of exfoliation occurring mid-treatment; if the probability of exfoliation occurring mid-treatment is greater than a threshold (mid-treatment eruption threshold), e.g., Z %, then the recommendation is for a shorter treatment.

In any of these methods and apparatuses, the probability analysis for any of these estimates (e.g., post-treatment eruption, pre-treatment eruption, mid-treatment eruption) may include: a strict probability of losing a tooth by age X, a probability of losing a tooth by age Y, and/or a probability of losing a tooth based on the current age and/or current dentition (conditional on dentition). The probability of losing a tooth by age Y may be a conditional tooth loss probability. For example, assuming that the patient hasn't lost the tooth by age X, which could include renormalizing the remaining probability of tooth loss (e.g., if cumulative probability of losing a tooth by age 11 is 0.6, but you haven't lost it yet, then one model of conditional probability is to renormalize the remaining probability by stretching it to 0-1).

Conditional probabilities may be determined using the patient database (e.g., a frequency count from the patient database), historical data, and/or a literature survey. In any of the methods and apparatuses described herein a hierarchical statistical model using Bayesian machine learning may be used. Alternatively or additionally, a black box model where a prediction is made using machine learning models may be used (e.g., random forest, decision trees, neural networks, etc.).

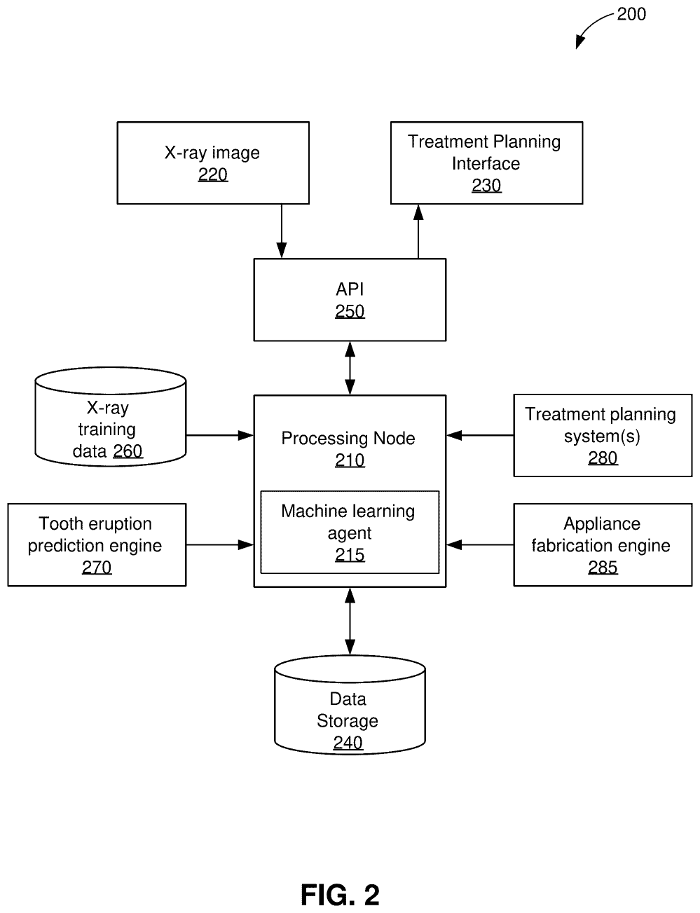

FIG. 2 schematically illustrates one example of a machine-learning tooth eruption prediction apparatus 200. Although described herein as a system, the machine-learning tooth eruption prediction apparatus 200 may be realized with any feasible apparatus, e.g., device, system, etc., including hardware, software, and/or firmware. In some examples, the machine-learning tooth eruption prediction apparatus 200 may include a processing node 210, an application programming interface (API) 250, and a data storage module 240. As shown, the API 250 and the data storage module 240 may each be coupled to the processing node 210. In some examples, all components of the machine-learning tooth eruption prediction apparatus 200 may be realized as a single device (e.g., within a single housing). In some other examples, components of the machine-learning tooth eruption prediction apparatus 200 may be distributed within separate devices. For example, the coupling between any two or more devices, nodes (either of which may be referred to herein as modules), and/or data storage modules may be through a network, including the Internet. In this manner, the machine-learning tooth eruption prediction apparatus 200 may be configured to operate as a cloud-based apparatus where some or all of the components of the machine-learning tooth eruption prediction apparatus 200 may be coupled together through any feasible wired or wireless network, including the Internet.

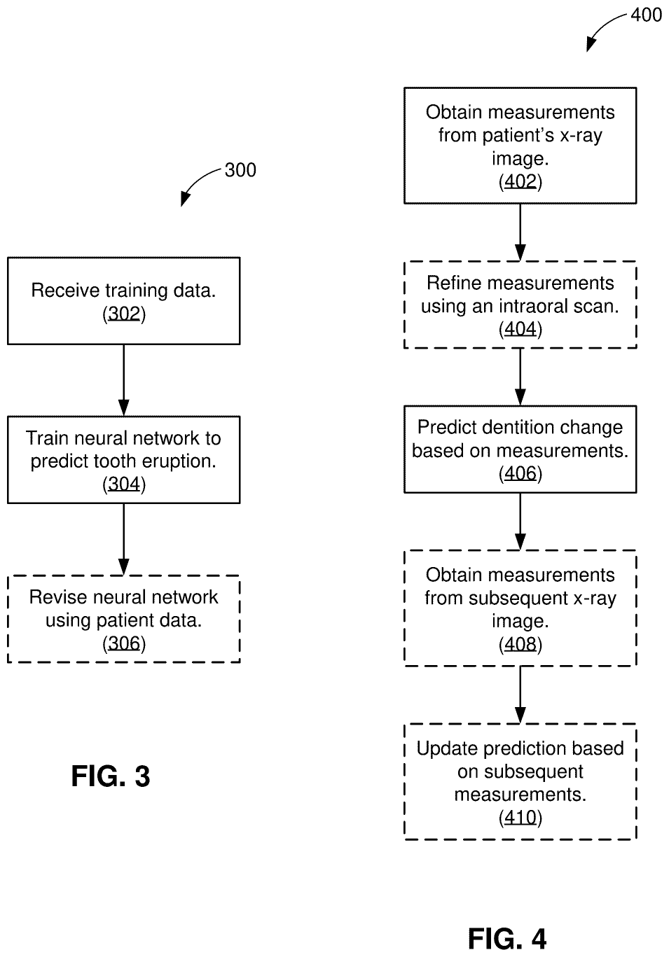

The machine-learning tooth eruption prediction apparatus 200 may predict one or more tooth eruptions associated with a patient based on information associated with or determined from the patient's x-ray images. In some embodiments, the machine-learning tooth eruption prediction apparatus 200 may use the API 250 to facilitate the receiving or input of patient x-ray images 220 and the outputting of treatment data through a treatment planning interface 230. The machine-learning tooth eruption prediction apparatus 200 may also include a tooth eruption prediction engine 270, a treatment planning system(s) 280, and an appliance fabrication engine 285. The tooth eruption prediction engine 270 may predict the eruption of one or more patient teeth based on patient x-ray images. In some variations, the tooth eruption prediction engine 270 may perform machine learning, including executing one or more neural networks trained to predict tooth eruption using a patient's x-ray images. For example, the processing node 210 (and/or the machine learning agent 215) may use x-ray training data 260 to train one or more neural networks that may form all or part of the tooth eruption prediction engine 270. In some variations, the processing node 210 may provide patient x-ray images 220 to the tooth eruption prediction engine 270. The tooth eruption prediction engine 270 may, in turn, predict tooth eruptions based, at least in part, on dental characteristics (e.g., tooth measurements) determined from the patient x-ray images 220. Training of the neural network is described in more detail in conjunction with FIG. 3.

The treatment planning system(s) 280 may generate, create, and/or provide any feasible dental or orthodontic treatment plans for a patient. The treatment planning system(s) 280 may receive or obtain patient data and treatment preferences relevant to a user. As described herein, the treatment planning interface 230 can transmit and/or receive patient data, user treatment preferences, as well as dental appliance data. Thus, using the patient data and user treatment preferences, the treatment planning system(s) 280 can generate or provide the associated treatment plans. The treatment planning system(s) 280 may implement automated and/or real-time treatment planning.

The treatment planning system(s) 280 may include one or more engines configured to generate and/or provide treatment plans. In various implementations, the treatment planning system(s) 280 identify and/or calculate treatment plans with instructions to treat medical conditions. The treatment plans may specify treatment goals, specific outcomes, intermediate outcomes, and/or recommended appliances used to achieve goals/outcomes. The treatment plan may also include treatment lengths and/or milestones. In various implementations, the treatment planning system(s) 280 calculate orthodontic treatment plans to treat malocclusions of teeth, restorative treatment plans for a patient's dentition, medical treatment plans, etc. The treatment plan may comprise automated and/or real-time elements and may include techniques described in U.S. patent application Ser. No. 16/178,491, entitled “Automated Treatment Planning.” In various implementations, the treatment planning system(s) 280 are managed by treatment technicians. As noted herein, the treatment plans may accommodate patient data in light of treatment preferences of users.

In some examples, the treatment planning systems(s) 280 may generate appliance data based on a patient's treatment plan. In some examples, a patient's appliance data may describe a series of sequential aligners that may be used to execute (perform or provide) a patient's treatment plan.

The treatment planning system(s) 280 may include engines that allow users to visualize, interact with, and/or fabricate appliances that implement a treatment plan. The treatment planning system(s) 280 may support UIs that display virtual representations of orthodontic appliances that move a patient's teeth from an initial position toward a final position to correct malocclusions of teeth. The treatment planning system(s) 280 can similarly include engines that enable the display of representations of restorative appliances and/or other medical appliances. The treatment planning system(s) 280 may support fabrication of appliances through, e.g., the appliance fabrication engine 285. The treatment planning system(s) 280 may also include engines to support user interaction with treatment plans. In some variations, treatment templates may include structured data, UI elements (forms, text boxes, UI buttons, selectable UI elements, etc.), etc.

The treatment planning system(s) 280 may include one or more data stores configured to store treatment templates expressed according to treatment domain-specific protocols. In some variations, the treatment templates may be stored in the data storage module 240.

The appliance fabrication engine 285 may be configured to fabricate one or more appliances, including dental and non-dental appliances. Examples of dental appliances include aligners, other polymeric dental appliances, crowns, veneers, bridges, retainers, dental surgical guides, etc. Examples of non-dental appliances include orthotic devices, hearing aids, surgical guides, medical implants, etc.

The appliance fabrication engine 285 may comprise thermoforming systems configured to indirectly and/or directly form appliances. The appliance fabrication engine 285 may implement instructions to indirectly fabricate appliances. As an example, the appliance fabrication engine 285 may be configured to thermoform appliances over a positive or negative mold. Indirect fabrication of a dental appliance can involve one or more of the following steps: producing a positive or negative mold of the patient's dentition in a target arrangement (e.g., by additive manufacturing, milling, etc.), thermoforming one or more sheets of material over the mold in order to generate an appliance shell, forming one or more structures in the shell (e.g., by cutting, etching, etc.), and/or coupling one or more components to the shell (e.g., by extrusion, additive manufacturing, spraying, thermoforming, adhesives, bonding, fasteners, etc.). Optionally, one or more auxiliary appliance components as described herein (e.g., elastics, wires, springs, bars, arch expanders, palatal expanders, twin blocks, occlusal blocks, bite ramps, mandibular advancement splints, bite plates, pontics, hooks, brackets, headgear tubes, bumper tubes, palatal bars, frameworks, pin-and-tube apparatuses, buccal shields, buccinator bows, wire shields, lingual flanges and pads, lip pads or bumpers, protrusions, divots, etc.) are formed separately from and coupled to the appliance shell (e.g., via adhesives, bonding, fasteners, mounting features, etc.) after the shell has been fabricated.

The appliance fabrication engine 285 may comprise direct fabrication systems configured to directly fabricate appliances. As an example, the appliance fabrication engine 285 may include systems, devices, or apparatuses configured to use additive manufacturing techniques (also referred to herein as “3D printing”) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can include: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliance fabrication engine 170 may be configured to directly fabricate appliances using selective laser sintering. In some embodiments, selective laser sintering involves using a laser beam to selectively melt and fuse a layer of powdered material according to a desired cross-sectional shape in order to build up the object geometry. As yet another example, the appliance fabrication engine 285 may be configured to directly fabricate appliances by fused deposition modeling. In some embodiments, fused deposition modeling involves melting and selectively depositing a thin filament of thermoplastic polymer in a layer-by-layer manner in order to form an object. In yet another example, the appliance fabrication engine 285 may be configured to implement material jetting to directly fabricate appliances. In some embodiments, material jetting involves jetting or extruding one or more materials onto a build surface in order to form successive layers of the object geometry.

In some embodiments, the appliance fabrication engine 285 may include a combination of direct and indirect fabrication systems. In some embodiments, an appliance fabrication system(s) (not shown) may be configured to build up object geometry in a layer-by-layer fashion, with successive layers being formed in discrete build steps. Alternatively or in combination, the appliance fabrication engine 285 may be configured to use a continuous build-up of an object's geometry, referred to herein as “continuous direct fabrication.” Various types of continuous direct fabrication systems can be used. As an example, in some embodiments, the appliance fabrication engine 285 may use “continuous liquid interphase printing,” in which an object is continuously built up from a reservoir of photopolymerizable resin by forming a gradient of partially cured resin between the building surface of the object and a polymerization-inhibited “dead zone.” In some embodiments, a semi-permeable membrane is used to control transport of a photopolymerization inhibitor (e.g., oxygen) into the dead zone in order to form the polymerization gradient. Examples of continuous liquid interphase printing systems are described in U.S. Patent Publication Nos. 2015/0097315, 2015/0097316, and 2015/0102532, (corresponding to U.S. Pat. Nos. corresponding to U.S. Pat. Nos. 9,205,601, 9,216,546, and 9,211,678) the disclosures of each of which are incorporated herein by reference in their entirety. As another example, the appliance fabrication engine 285 may be configured to achieve continuous build-up of an object geometry by continuous movement of the build platform (e.g., along the vertical or Z-direction) during the irradiation phase, such that the hardening depth of the irradiated photopolymer is controlled by the movement speed. Accordingly, continuous polymerization of material on the build surface can be achieved. Example systems are described in U.S. Pat. No. 7,892,474, the disclosure of which is incorporated herein by reference in its entirety.

In another example, the appliance fabrication engine 285 may be configured to extrude a composite material composed of a curable liquid material surrounding a solid strand. The composite material can be extruded along a continuous 3D path in order to form the object. Examples systems are described in U.S. Patent Publication No. 2014/0061974, corresponding to U.S. Pat. No. 9,511,543, the disclosures of which are incorporated herein by reference in its entirety.

In yet another example, the appliance fabrication engine 285 may implement a “heliolithography” approach in which a liquid photopolymer is cured with focused radiation while the build platform is continuously rotated and raised. Accordingly, the object geometry can be continuously built up along a spiral build path. Examples of such systems are described in U.S. Patent Publication No. 2014/0265034, corresponding to U.S. Pat. No. 9,321,215, the disclosures of which are incorporated herein by reference in its entirety.

The data storage module 240 may be any feasible data storage unit, device, structure, including random access memory, solid state memory, disk-based memory, non-volatile memory, and the like. The data storage module 240 may store image data, including patient x-ray images 220 received through the API 250. The data storage module 240 may also store appliance data from the appliance fabrication engine 285.

The data storage module 240 and/or the processing node 210 may also include a non-transitory computer-readable storage medium stores instructions that may be executed by the processing node 210. For example, the processing node 210 may include one or more processors (not shown) that execute instructions stored in the data storage module 240 to perform any number of operations including operations for assessing the patient x-ray images 220 and predicting tooth eruption. For example, the data storage module 240 may store one or more neural networks that may be trained and/or executed by the processing node 210. Alternatively, the processing node 210 may include one or more machine-learning agents 215 (e.g., trained neural networks, as described herein), as shown in FIG. 1.

In some examples, the data storage module 240 may include instructions to train one or more neural networks to assess patient x-ray images 220. More detail regarding training of the neural networks are described below in conjunction with FIG. 3. Additionally, or alternatively, the data storage module 240 may include instructions to execute one or more neural networks to assess the patient x-ray images 220. More detail regarding the execution of a neural network is described below.

FIG. 3 is a flowchart showing an example method 300 for training a neural network to predict tooth eruption. Some examples may perform the operations described herein with additional operations, fewer operations, operations in a different order, operations in parallel, and some operations differently. The method 300 is described below with respect to the machine-learning tooth eruption prediction apparatus 200 of FIG. 2, however, the method 300 may be performed by any other suitable apparatus, system, or device. In some examples, the neural network may be trained to predict tooth eruption from a patient's x-ray image and/or associated dental measurements.

The method 300 begins in block 302 as the processing node 210 obtains x-ray training data 260. The x-ray training data 260 may include dental x-ray images (e.g., dental images) that show one or more aspects of a patient's dentition. The x-ray images may include multiple bitewing x-rays and, in some cases, panoramic x-ray images. For example, the x-ray training data 260 may include x-ray images of some or all of a person's teeth, soft tissue, bone structure, etc. The x-ray training data 260 may also include data, especially measurement data, associated with each x-ray images. For example, when the x-ray training data 260 includes teeth that have not yet erupted, the x-ray training data 260 may also include measurement data associated with those teeth. The measurement data may include crown to gingival distance and/or a relative distance between an unerupted tooth and a medial tooth's crown for any unerupted teeth, as well as tooth number. The x-ray training data 260 may also include patient demographic data including, but not limited to age, gender, weight, ethnicity, geographic location and the like. Any of the x-ray training data 260 images described herein may include panoramic x-ray images, bitewing x-ray images, periapical x-ray images, or a combination thereof.

Next, in block 304 the processing node 210 may train one or more neural networks to predict tooth eruption based on an input x-ray image. In some examples, the input x-ray image may also include patient data such as patient age, gender, weight, ethnic background, geographic location, and the like. In some examples, the input x-ray image may also include tooth measurement data that may be determined from the input x-ray image. As an example, the input x-ray image may include measurement data of crown-to-gingival distances for unerupted teeth.

The processing node 210 can train a variety of neurons to recognize various aspects of the x-ray training data 260 and predict associated tooth eruption times, as well as other tooth data such as component variations in erupting tooth geometry. In some examples, the processing node 210 may execute or perform any feasible supervised or unsupervised learning algorithm to train the neural network. For example, the processing node 210 may execute linear classifiers, support vector machines, decision trees or algorithms to predict tooth eruptions from all available data associated with an input x-ray image.

In some other examples, the processing node 210 may execute or perform any feasible regression algorithm (including linear regression models) to train the neural network to predict tooth eruption and/or to determine any associated erupting tooth geometry.

In some variations, prior to training any neural network the processing node 210 may adjust a contrast or brightness associated with any images of the x-ray training data 260. Adjustment of the contrast or brightness may enable the processing node 210 to more easily detect any dental features (e.g., teeth, gingiva, soft tissue, etc.) in the x-ray training data 260. The trained neural networks may be stored in the data storage module 240.

Next, in block 306 the processing node 210 can revise the training of the neural network using patient data. This step may be optional, as indicated with dashed lines in FIG. 3. As an example, using the neural network, a clinician can predict (at time T₁) an eruption time of a first tooth. An additional patient x-ray may be obtained that is performed at a later time T₂ (T₂ is after T₁). Measurements associated with the first tooth (and any other tooth in the process or erupting or exfoliating) may be obtained from the additional patient x-ray. Using these measurements, the trained neural network may be updated to provide more accurate predictions of the patient's tooth eruptions. In some variations, a fitted slope may be used to determine the first tooth's eruption speed. The neural network may then be updated to use data associated with the first tooth's eruption speed to predict tooth eruption.

FIG. 4 is a flowchart showing one example of a method 400 for predicting tooth eruption for a patient using the machine learning approach of FIG. 3. The method 400 is described below with respect to the machine-learning tooth eruption prediction apparatus 200 of FIG. 2 however, the method 400 may be performed by any other suitable apparatus, system, or device.

The method 400 begins in block 402 as the processing node 210 obtains or determines dental measurements from an x-ray image 220 of a patient. As an example, the x-ray image 220 may include one or more panoramic x-ray images of the patient. In some other examples, the x-ray image 220 may include one or more bitewing or periapical x-rays of the patient. In some variations, the processing node 210 may adjust a contrast or brightness associated with any x-ray image 220 of the patient. Adjustment of the contrast or brightness may enable the processing node 210 to more easily detect any dental features (e.g., teeth, gingiva, soft tissue, etc.) in the x-ray images of the patient. The dental measurements may be determined by measuring the distance between features using the x-ray image 220. In some examples, the dental measurements may include crown to gingival distance for any unerupted teeth, as well as the associated tooth number.

Next, in block 404 dental measurement may be refined using an intraoral scan. For example, in some instances, a panoramic x-ray may show one or more erupting teeth, however the panoramic x-ray may lack sufficient detail to obtain dental measurements. A corresponding intraoral scan may be used to determine the dental measurement of any feasible teeth that may be used to estimate tooth eruption. This step may be optional, for example, when the panoramic x-rays include sufficient detail, block 404 may be skipped.

Next, in block 406 the processing node 210 predicts tooth eruption based on the dental measurements determined from the patient's x-rays. For example, the processing node 210 may execute one or more machine learning-based programs and/or neural networks to predict tooth eruption. Some neural networks may be trained as described with respect to FIG. 3. The predicted tooth eruptions may provide a probability of exfoliation in terms of weeks or months from a reference time. The reference time may be associated with a time that the patient's x-ray scan was captured.

Next, in block 408 the processing node 210 may obtain measurements from a patient's subsequent x-ray scan. The measurements obtained in block 408 may be similar to those described in blocks 402 and 404. However, these measurements are based on a later x-ray scan. Using two or more time-separated x-ray scans may enable a more accurate prediction of tooth eruption and/or exfoliation. In some embodiments, block 408 may be optional.

Next, in block 410, the processing node 210 updates the eruption prediction based on the subsequent measurements determined in block 408. As described with respect to FIG. 3, a tooth eruption or exfoliation prediction may be updated based on a second, more recent set of dental measurements.

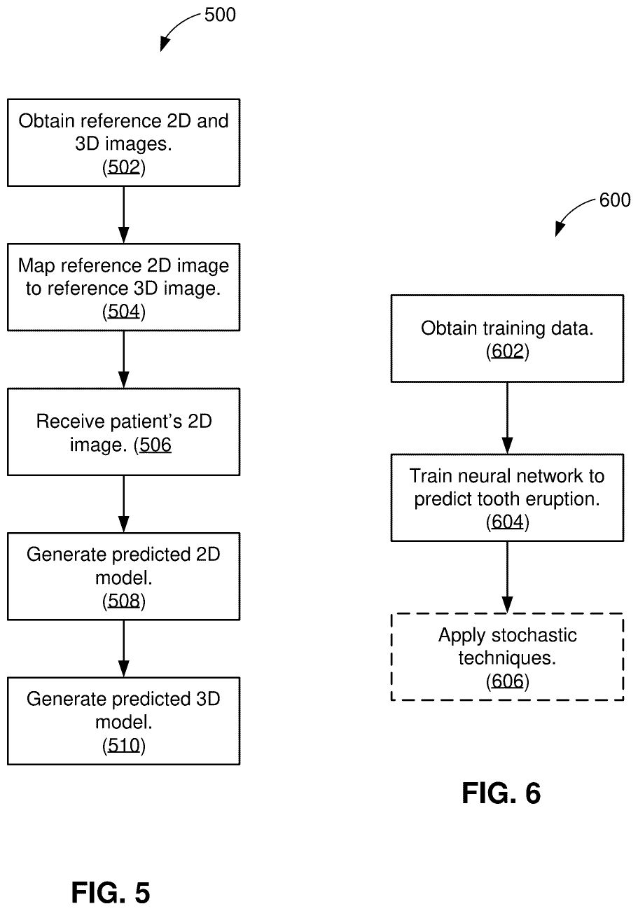

FIG. 5 is a flowchart showing one example of a method 500 for predicting tooth eruption location and direction for a patient. The method 500 is described below with respect to the machine-learning tooth eruption prediction apparatus 200 of FIG. 2, however, the method 500 may be performed by any other suitable apparatus, system, or device. Generally, two-dimensional (2D) x-ray images are easier to obtain than 3D cone-beam computed tomography (CBCT) or conventional computer tomography (CT) images in dental clinics. Furthermore, 2D x-ray images may also have the advantage of low x-ray dosage. However, it may be difficult to determine the 3D information, especially the direction and location of tooth eruption solely from 2D x-ray images. Described herein is a method to update 2D x-ray information with 3D-based image data to improve tooth eruption and exfoliation prediction.

The method 500 begins in block 502 as the processing node 210 obtains or receives a reference 2D image and a related reference 3D image. For example, the reference 2D image and the reference 3D image may be of the same person or model. Therefore, the teeth, gums, and other dentals anatomies of the two reference images may share a one-to-one correspondence. Additionally, the reference 2D image and the reference 3D image may be prepared or formed into an “ideal arch.” The ideal arch may describe idea positions of teeth within the arch. Different ideal arches may exist for different patient's ages, gender, weight, or any other feasible demographic information.

In some examples, the 2D image may be a panoramic x-ray image, a series of bitewing x-rays, or any other feasible 2D x-ray image. The 3D image may be a CBCT scan, a CT scan, or any other feasible 3D x-ray image.

Next, in block 504 the processing node 210 maps the reference 2D image to the reference 3D image. In other words, elements of the reference 2D image may be mapped or morphed to the same elements in the reference 3D image. The morphing (mapping) performed in block 504 may be used to establish a transformation function between 2D images and related 3D images.

Next, in block 506 the processing node 210 receives (or obtains) a patient's 2D x-ray image. The patient's 2D x-ray image may be a panoramic x-ray, a series of bitewing x-rays, or any other feasible 2D x-ray image. The patient's 2D x-ray image may represent a starting point for a patient to receive a dental treatment.

Next, in block 508 the processing node 210 generates a predicted 2D dental model. For example, the processing node 210 may morph (map) the patient's 2D x-ray image (received in block 506) to the reference 2D x-ray image. In some embodiments, the morphing may map teeth or other dental anatomies in the patient's 2D x-ray to like teeth and dental anatomies in the reference 2D image.

Next, in block 510 the processing node 210 generates a predicted 3D dental model. The processing node morphs (maps) the 2D dental prediction of block 508 into a predicted 3D model. In some embodiments, the processing node 210 can use a similar transformation function as established in block 504 to morph the 2D model to the 3D model. Using the predicted 3D dental model, a clinician can predict tooth eruption and/or exfoliation for any patient. For example, the predicted 3D dental model may provide a direction and location for erupting and/or exfoliating teeth.

In some cases, the erupted teeth in a panoramic 2D image could be mapped to a patient intraoral scan, such as iTero scan, and then use that reference to address unerupted teeth. In the absence of 2D panoramic or CBCT images, the geometry of the unerupted tooth can be predicted using statistical shape modelling in which the tooth geometry is predicted based on the principal component variations in tooth geometry for each individual tooth. Variation in tooth geometry for each tooth can be broken into principal variation modes (e.g. crown width, intercusp distance, molar groove depth). Each mode corresponds to a certain percentage of variation in tooth geometry among the population, and so using this method, a tooth geometry which can capture the largest portion of the population can be calculated and utilized in the aligner.

The position of a patient's head and scanning parameters when taking any x-ray images may affect the accuracy of the predicted model. In some cases, a user could physically measure the tooth size (for example width of incisor) and then scale the scanned images. The scanning should also follow the protocol, and use same scanning setup every time for model accuracy.

In some variations, tooth eruption, exfoliation, and or placement may be predicted based on one or more facial features of a patient. For example, a machine learning neural network may be trained to predict the morphology of erupted and exfoliated teeth. An example of this method is described below in conjunction with FIGS. 6-8.

FIG. 6 is a flowchart showing an example method 600 for training a neural network to predict tooth eruption based on facial features. Although described using facial features herein, the neural network may be trained to predict tooth eruption based on any other feasible physical features or characteristics. The method 600 is described below with respect to the machine-learning tooth eruption prediction apparatus 200 of FIG. 2, however, the method 600 may be performed by any other suitable apparatus, system, or device.

The method 600 begins in block 602 as the processing node 210 obtains or receives training data. The training data may include facial feature data as well as x-ray image data. The facial feature data may include any feasible location and measurement information of various facial features or landmarks. For example, facial features may include jaw width, head size, jaw height, and the like. The x-ray image data may include 2D and/or 3D x-ray images as well as any measurement data that may be associated with each of the x-ray images. The measurement data may include crown to gingival distance for any unerupted teeth, tooth number, tooth orientation, and the like.

Next, in block 604 the processing node 210 may train one or more neural networks to predict tooth eruption and/or exfoliation based on facial features. As described above, the facial features may include any feasible facial features, landmarks, as well as any associated or related measurements. For example, the processing node 210 may construct one or more linear regression models, tree-based models, and the like to train one or more neural networks to predict teeth morphology. Tooth morphology may include the length, height, and width of a bounding box, and other tooth parameters like crown height and circumference.

Next, in block 606 the processing node 210 may apply stochastic techniques to the outcomes or predictions of the trained neural network. For example, various prediction models may predict different tooth geometries. It is vital that any predictive model takes individual variances among patients into consideration.

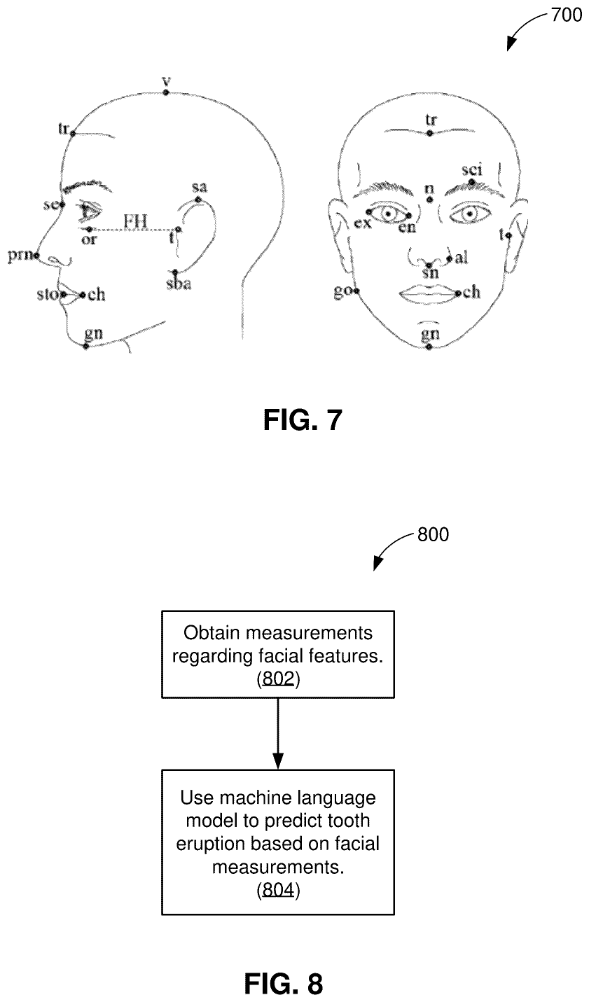

FIG. 7 shows an illustration of a face 700 with the location of possible facial datums. In some instances, measurements between these datums may be made as part of the facial feature data described in FIG. 6. Although one example of facial datums are shown in FIG. 6, in other variations, any number of similar or different facial datums may be used.

FIG. 8 is a flowchart showing one example of a method 800 for predicting tooth eruption for a patient using the machine learning approach of FIG. 6. The method 800 is described below with respect to the machine-learning tooth eruption prediction apparatus 200 of FIG. 2 however, the method 800 may be performed by any other suitable apparatus, system, or device.

The method begins in block 802 as the processing node 210 obtains or determines facial measurements of a patient. The facial measurements may be input by a user or may be determined by a processor (such as the processing node 210) analyzing one or more facial photographs.

Next, in block 804 the processing node 210 uses a machine language mode to predict tooth eruption based on facial measurements. For example, the processing node 210 may execute one or more machine learning-based programs and/or neural networks to predict tooth eruption. Some neural networks may be trained as described with respect to FIG. 6. The predicted tooth eruptions may provide a probability of exfoliation in terms of weeks or months from a reference time.

After determining an estimate of tooth eruption and/or exfoliation, a clinician may create or modify a treatment plan that considers the change of the patient's dentition.

### Treatment Planning Timing

In some cases, the clinician may be able to change treatment times to accommodate a tooth eruption and/or exfoliation. For example, if predictions indicate that a baby tooth may exfoliate within a month, the clinician may decide to delay beginning treatment by about a month.

### Primary Tooth Extraction

In some cases, the clinician may decide to extract the primary tooth (baby tooth) prior to commencement of the treatment plan. The tooth eruption/exfoliation predictions may enable the clinician to determine when a tooth eruption/exfoliation may occur with respect to a treatment plan. For example, if a tooth is predicted to erupt in the middle of a treatment plan, then the clinician may decide to remove the tooth prior to starting a treatment. Furthermore, some studies may show that baby teeth may exfoliate more rapidly when a patient is undergoing dental aligner treatment. Thus, it may be advantageous to remove baby teeth prior to beginning a treatment plan that includes wearing a dental aligner.

### Aligner Modifications

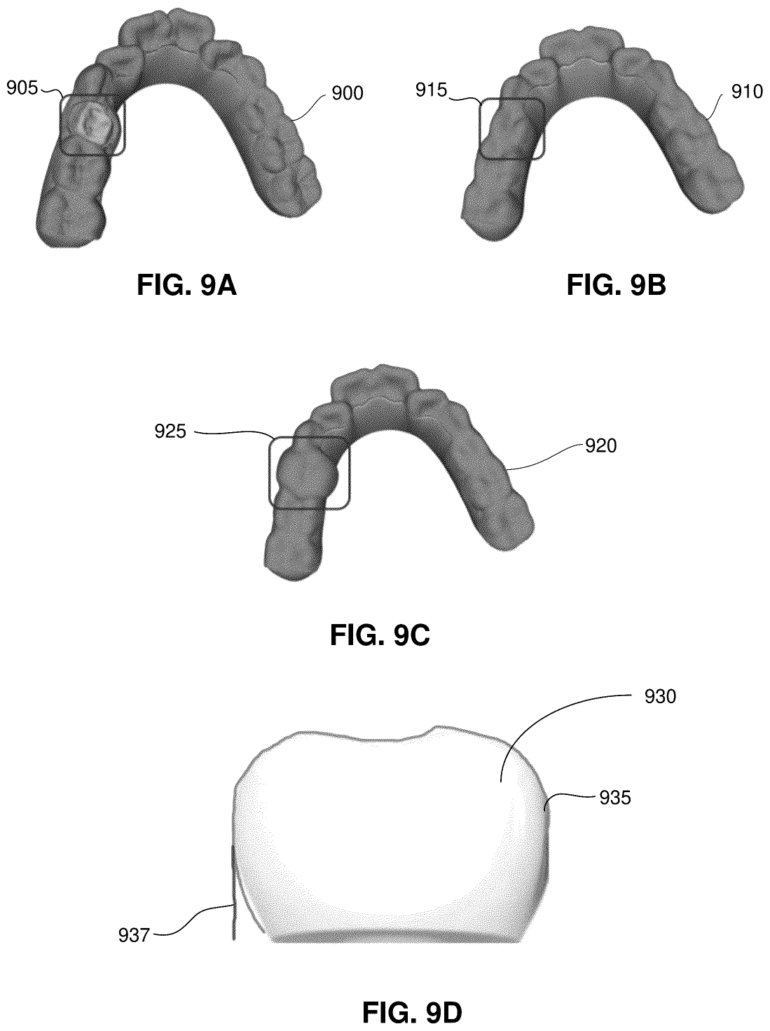

Knowing a predicted tooth eruption/exfoliation time may enable the clinician to modify the treatment plan accordingly. For example, one or more dental aligners may be designed to accommodate an erupting tooth. FIGS. 9A-9D show example dental aligners that are designed to accommodate an erupting tooth. FIG. 9A shows a dental aligner 900 that includes a cut-out region 905. The cut-out region 905 is centered around a location of a baby tooth predicted to be exfoliated. In this manner, the cut-out region 905 can accommodate a patient's dentition before and after an eruption of a permanent tooth.

FIG. 9B shows a primary dental aligner 910 that may be designed according to the dentition geometry of primary teeth, in particular before an eruption of a permanent tooth. Thus, the primary dental aligner 910 includes a conventional tooth pocket 915 for the non-erupted tooth. FIG. 9C shows an eruption-compensated aligner 920 that is designed in accordance with a predicted position of an erupted permanent tooth. The eruption-compensated aligner 930 may include an eruption-compensated pocket 925 that may be larger than a conventional tooth pocket 915. The eruption-compensated pocket 925 may minimize the risk of tooth impaction.

FIG. 9D shows another possible modification to a dental aligner 930 to accommodate an erupting tooth. In this example, the dental aligner 930 may include a pocket 935 (shown in profile) that includes an undercut region 937 that can accommodate movement associated with an erupting tooth.

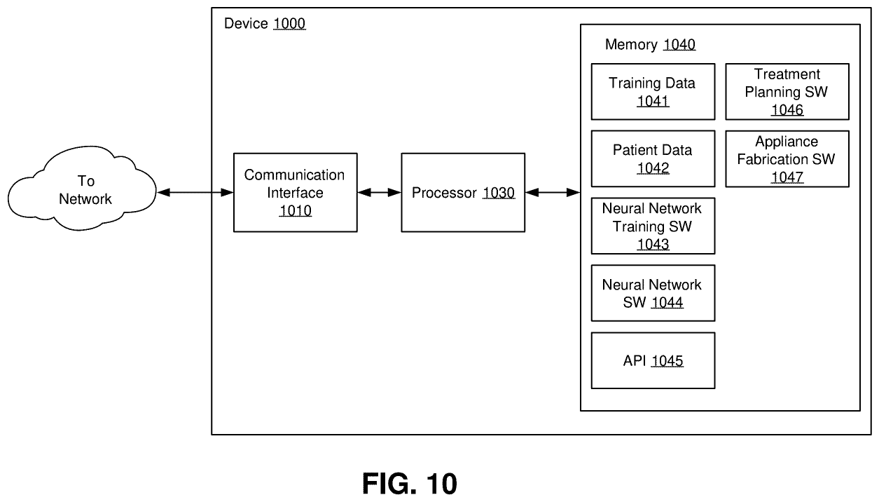

FIG. 10 shows a block diagram of a device 1000 that may be one example of the machine-learning tooth eruption prediction apparatus 200 of FIG. 2. Although described herein as a device, the functionality of the device 1000 may be performed by any feasible apparatus, system, or method. The device 1000 may include a communication interface 1010, a processor 1030, and a memory 1040.

The communication interface 1010, which may be coupled to a network and to the processor 1030, may transmit signals to and receive signals from other wired or wireless devices, including remote (e.g., cloud-based) storage devices, cameras, processors, compute nodes, processing nodes, computers, mobile devices (e.g., cellular phones, tablet computers and the like) and/or displays. For example, the communication interface 1010 may include wired (e.g., serial, ethernet, or the like) and/or wireless (Bluetooth, Wi-Fi, cellular, or the like) transceivers that may communicate with any other feasible device through any feasible network. In some examples, the communication interface 1010 may receive training data 1041 and/or patient data 1042.

The processor 1030, which is also coupled to the memory 1040, may be any one or more suitable processors capable of executing scripts or instructions of one or more software programs stored in the device 1000 (such as within memory 1040).

The memory 1040 may include training data 1041. The training data 1041 may include a plurality of 2D and/or 3D x-ray images that include associated patient characteristics. The x-ray images may include panoramic x-rays, bitewing x-rays, CT scans, CBCT scans, or the like. As described above, the patient characteristics may include any feasible measurement associated with any feasible dental anatomies. By way of example and not limitation, the dental characteristics may include crown to gingival distance for any tooth, as well as a tooth number identifying an individual tooth. In some variations, the patient characteristics may include patient demographic data including gender, age, weight, ethnic background, geographic location, and the like. In some examples, the training data may include facial features or datums as well as measurements associated with those facial features.

The memory 1040 may also include patient data 1042. The patient data 1042 may include one or more patient x-rays that are to be evaluated by the device 1000 to determine a prediction of an erupted tooth for a patient. In some examples, the patient data 1042 may include panoramic or bitewing x-rays. In some further examples, the patient data 1042 may also include a patient's facial image with two or more facial datums. The patient data 1042 may include measurements associated with the facial datums.

The memory 1040 may also include a non-transitory computer-readable storage medium (e.g., one or more nonvolatile memory elements, such as EPROM, EEPROM, Flash memory, a hard drive, etc.) that may store a neural network training software (SW) module 1043, a neural network SW module 1044, an API 1045, a treatment planning SW module 1046, and an appliance fabrication SW module 1047. Each software module includes program instructions that, when executed by the processor 1030, may cause the device 1000 to perform the corresponding function(s). Thus, the non-transitory computer-readable storage medium of memory 1040 may include instructions for performing all or a portion of the operations described herein

The processor 1030 may execute the neural network training SW module 1043 to train one or more neural networks to perform one or more of the operations discussed with respect to FIGS. 2-8. In some examples, execution of the neural network training SW module 1043 may cause the processor 1030 to collect or obtain training data (such as x-ray images, facial images, and associated patient characteristics within the training data 1041) and train a neural network using the training data 1041. The trained neural network may be stored as one or more neural networks in the neural network training SW module 1043.

The processor 1030 may execute one or more neural networks in the neural network SW module 1044 to assess patient x-ray images (which may be stored in the patient data 1042) to determine a prediction of a tooth eruption or exfoliation. For example, execution of a neural network may assess a patient x-ray and determine a prediction of a tooth eruption. In another example, execution of a neural network may assess an image of a patient's face in order to determine an estimate of tooth eruption.

The processor 1030 may execute instructions in the API 1045 to receive patient x-ray images that may be included with the patient data 1042 and output treatment recommendations that may be determined by executing the neural network SW module 1044. In some variations, the API 1045 may also output a treatment plan and/or dental appliance data.

The processor 1030 may execute the treatment planning SW module 1046 to generate and store a patient's treatment plan. In some examples, execution of the treatment planning SW module 1046 may also modify treatment preferences, generate modified treatment parameters, and/or generate a treatment plan based on predicted tooth eruption and/or exfoliation. Execution of the treatment planning SW module 1046 may access any applicable data (e.g., x-ray images, facial images, patient data, and the like) in the memory 1040.

The processor 1030 may execute the appliance fabrication SW module 1047 to generate aligner data that, in turn, may be used to fabricate one or more aligners. For example, execution of the appliance fabrication SW module 1047 may use patient data and treatment preferences stored in the memory 1040 to generate aligner data for a series of aligners.

### Examples

In some examples the methods and apparatuses described herein may estimate a prediction for eruption compensation, which may include one or more of: eruption timing, location, direction, and/or geometry. The predictions may be provided as an output directly, and/or they may be incorporated into a treatment planning, such as (but not limited to) orthodontic appliance designs that may incorporate the predicted information for treatment improvement.

In general, these methods and apparatuses may include time points as part of the eruption timing prediction, such as: t0, the earliest time point at which an extraction should be suggested; t1, the last moment before the primary-teeth-based treatment obstructs the tooth eruption; and t2, the time at which the erupted permanent tooth needs to be included in the treatment. The time tolerance for adjustment of/on treatments is typically from t0 to t1.

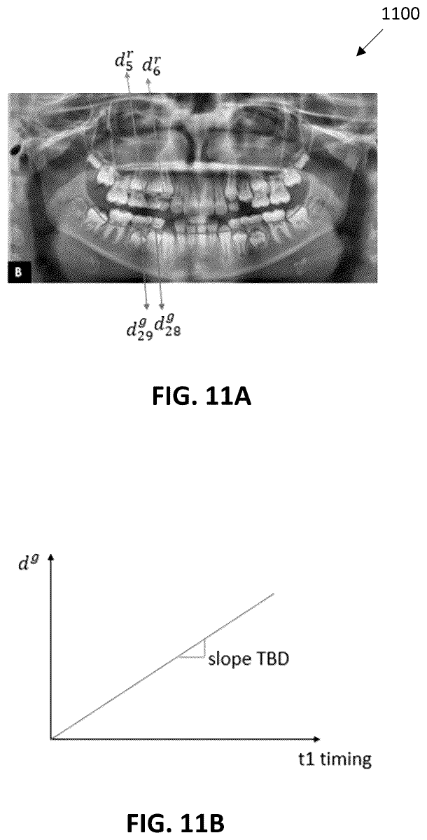

FIG. 11A illustrates a method of predicting tooth eruption based on an X-ray scan 1101. This example shows the relative distance between the crown to next medial tooth's crown for teeth 5 and 6 (on the upper jaw). Crown-to-gingiva distance for teeth 28 and 29 are shown on the lower jaw.

Using an image (e.g., x-ray image) such as that shown in FIG. 11A, the method or apparatus may determine a prediction based on image (e.g., X-ray scan) by first obtaining positions/distances (e.g., in mm) from the X-ray scan. Optionally, if the absolute distance is not clear in the image (e.g., X-ray scan), the image may be registered to an intraoral scan and then the tooth size in the scan may be used to get the absolute sizes in the image (e.g., X-ray scan). Based on this, the crown to gingival distance: de may be estimated. The crown to next medial tooth's crown (relative distance), d′ may also be estimated. The method or apparatus may follow the eruption order, and presume that the next possible tooth is erupting. Starting with this simple model, the method and/or apparatus may presume a linear relationship with distance and time, and may predict the t1 of the next erupting tooth by the measured distances. The method or apparatus may then predict the next erupting tooth by the relative distances. Any of these methods an apparatuses may calibrate the relationship between distance and time with existing data.

Any of the methods and apparatuses described herein may make one or more of these predictions based on rescan timing.

For example, if rescanning happens at t1 of the erupting tooth, then a linear regression machine-learning model may be trained and used to predict the next t1 with features such as gender, age, next possible likely erupting tooth. For example, a prediction of t0 and t2 based on t1 may include Modifying de with preset distance:

$$d = {d}^{g} - {d}_{0} \qquad t 0$$

$$d = {d}^{g} + {d}_{2} \qquad t 2$$

The method or apparatus may then use the fitted slope (e.g., speed) to determine the time, as illustrated in FIG. 11B. For example, using the present time gaps:

$$t 0 = t 1 - dt 0$$

$$t 2 = t 1 + dt 2$$

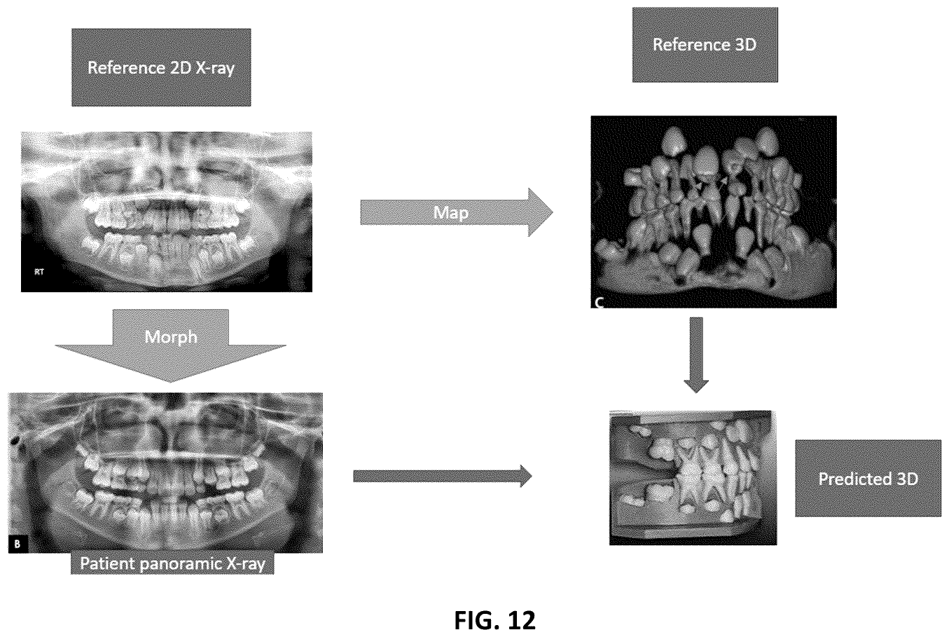

Also described herein are methods and apparatuses using x-ray images (scans) to predict erupting permanent teeth, including 3D location and direction. This is illustrated in FIG. 12, and was discussed above. An x-ray image may be easier to obtain than a CT images in dental clinics and may also have the advantage of lower X-ray dose. However, it is often difficult to get 3D information, especially the direction and location of tooth eruption. The methods and apparatuses described herein may predict permanent teeth 3D location and direction by mapping the 2D image to a reference 3D model (as shown in FIG. 12). As shown, a reference 2D X-ray and the according reference 3D CBCT/CT image may be prepared with an ideal arch. The X-ray could be panoramic or regular. The 2D panoramic view may be mapped to a 3D model of reference (e.g., ideal arch). The patient and the reference 2D images may then be morphed to show a patient 2D image. The same morphing to the ref 3D may be performed to provide a direction and location for the erupting teeth. Alternatively, the erupted teeth in the panoramic 2D image could be mapped to the patient intraoral scan, such as iTero scan, and then use that reference to address unerupted teeth.

In the absence of 2D panoramic or CBCT images, the geometry of the unerupted tooth can be predicted utilizing statistical shape modelling in which the tooth geometry is predicted based on the principal component variations in tooth geometry for each individual tooth. For example, variation in tooth geometry for each tooth can be broken into principal variation modes (e.g. crown width, intercusp distance, molar groove depth). Each mode may correspond to a certain percentage of variation in tooth geometry among the population, and so using this method, a tooth geometry which can capture the largest portion of the population can be calculated and utilized in the aligner. This analysis may be performed on tooth 3 from 10 random dentitions. The geometry mode shown in the video represents 32 percent of all tooth geometry variation in the dataset. Utilizing this form of analysis can permit tooth geometry prediction based on tooth position, and can further be stratified by patient demographic data to provide more accurate predictions.

The position of patient's head and scanning parameter during taking the X-ray image may affect the accuracy of the model. One could physically measure the tooth size (for example width of incisor) and then scale the scanned images. The scanning should also follow the protocol, and use the same scanning setup every time for model accuracy.

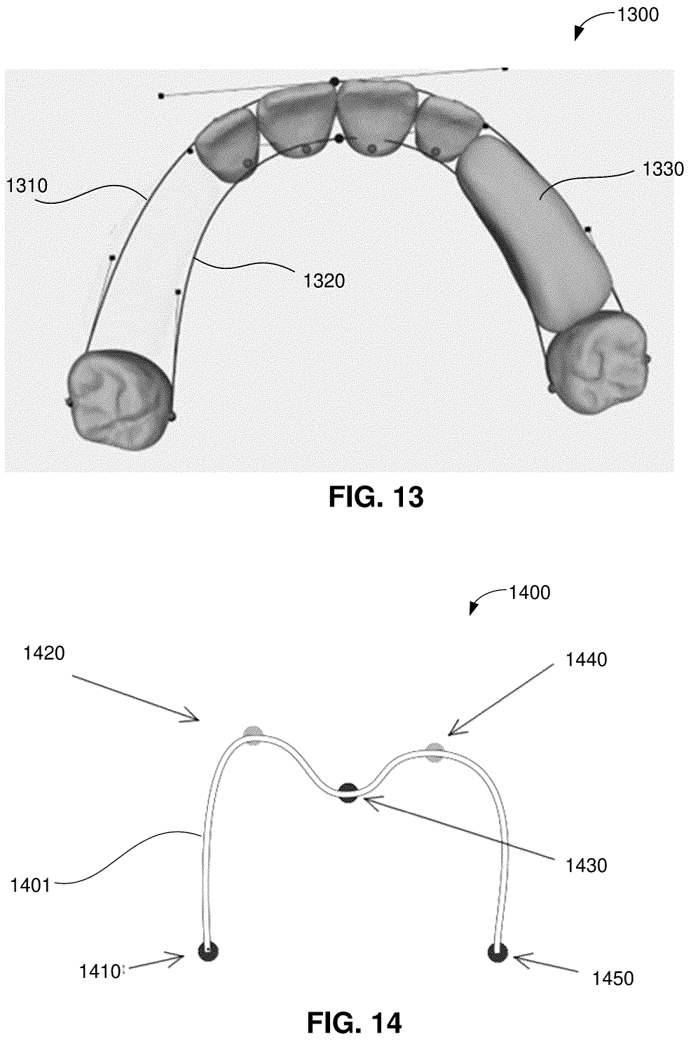

FIG. 13 shows a simplified dental arch 1300 illustrating another approach to accommodate unerupted teeth for aligner designs. An aligner may include a pocket, void, or “bubble region” that can receive one or more teeth as they erupt and grow into a dental arch. The bubble region may be an area that is generally open to allow a tooth to erupt unimpeded. Thus, a precise or accurate prediction regarding a tooth eruption (size, direction, placement) may not be necessary. The dental arch 1300, as drawn, may be associated with an upper jaw; however, in other examples, the dental arch 1300 may be associated with a lower jaw.

The example dental arch 1300 includes a bubble region 1330 (sometimes referred to as a void). As described above, the bubble region 1330 may be a portion of the dental arch 1300 that can accept one or more erupting teeth. Although the bubble region 1330 as shown can accommodate multiple erupting teeth, in other implementations, the bubble region 1330 can be sized to accommodate just a single tooth. The bubble region 1330 may provide sufficient room for teeth to erupt and grow without damaging or impacting a dental aligner.

In general, the bubble region 1330 can be defined, at least in part, by jaw arch splines. As shown, the dental arch 1300 may include a buccal jaw arch spline 1310 and a lingual jaw arch spline 1320. In general, a jaw arch spline may be described by a piecewise continuous polynomial that describes a curve passing through any predetermined points associated with the patient's teeth. The bubble region 1330 may fit between the buccal dental arch spline 1310 and the lingual dental arch spline 1320.

In some implementations, the bubble region 1330 can include details (e.g., shapes) regarding possible tooth profiles for the erupting teeth. In particular, tooth data from one or more of the patient's actual teeth may be used to provide additional jaw spline information for tooth profiles in the bubble region 1330. This is described in more detail below in conjunction with FIGS. 14-17.

FIG. 14 shows an example buccal-lingual plane 1400. A bubble region (such as the bubble region 1330 of FIG. 13) may be defined or determined by one or more buccal-lingual planes. Each buccal-lingual plane 1400 may be normal to a dental arch. Furthermore, each buccal-lingual plane 1400 may include a curve 1401 that may be determined by a profile of one or more existing teeth in the dental arch. In some embodiments, the curve 1401 may predict possible two-dimensional (2D) tooth profiles for teeth that can erupt into the bubble region 1330.

The curve 1401 may include five points that define a shape of a 2D tooth profile. In some variations, the curve 1401 may include fewer than five points and in still other variations, the curve 1401 may include more than five points. In one example, the curve 1401 may include a buccal gingiva point 1410, a buccal cusp point 1420, a groove point 1430, a lingual cusp point 1440, and a lingual gingiva point 1450. Each point 1410, 1420, 1430, 1440, and 1450 may be associated with any feasible characteristics or features of a patient's teeth.

In some examples, the points on the curve 1401 may be determined from jaw arch splines that are associated with particular points on the existing teeth of the patient. For example, the buccal gingiva point 1410 and the lingual gingiva point 1450 may be associated with jaw arch splines that are associated with regions where a tooth meets a region of the patient's gingiva.

The buccal cusp point 1420 and the lingual cusp point 1440 may be associated with jaw arch splines that pass through relatively high points on upper teeth surfaces. Thus, the buccal cusp point 1420 may be associated with a high point on a surface of a tooth located toward the patient's buccal side. The lingual cusp point 1440 may be associated with a high point on a surface of a tooth located toward the patient's lingual side. The groove point 1430 may be associated with a jaw arch spline that passes through a local minimum point on a surface of a tooth between the buccal cusp point 1420 and the lingual cusp point 1440.

In order to determine the points 1410, 1420, 1430, 1440, and 1450, the associated jaw arch splines may be projected through the buccal-lingual plane 1400. Where the jaw arch splines intersect the buccal-lingual plane 1400, the associated points are defined. For example, a buccal gingiva jaw arch spline may intersect the buccal-lingual plane 1400 at point 1410. Other points may be determined by the intersection of other jaw arch splines and the buccal-lingual plane 1400. The curve 1401 is determined by connecting the points 1410, 1420, 1430, 1440, and 1450. In some variations, the curve 1401 may be shaped with any feasible curve fitting operation. In other variations, the curve 1401 may be a piecewise linear curve (not shown).

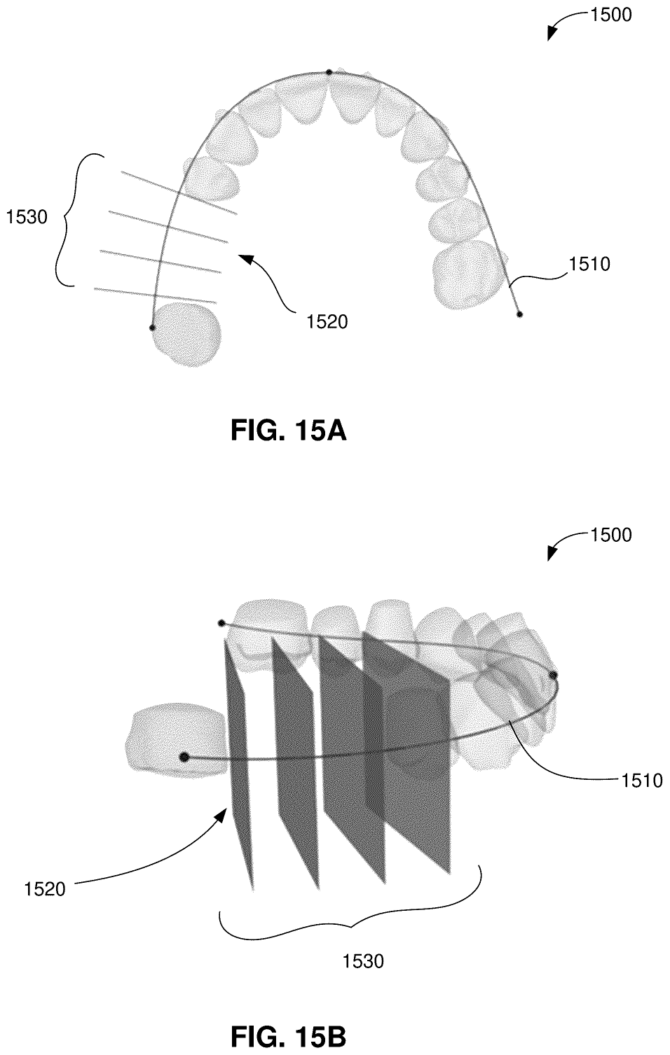

FIGS. 15A and 15B illustrate an example usage of buccal-lingual planes and jaw arch splines. FIG. 15A shows a dental arch 1500. As shown, the dental arch 1500 may be missing several teeth in region 1520. For example, the missing teeth may be unerupted teeth in a younger patient. Therefore, a dental aligner for the dental arch 1500 may need a void or bubble region in region 1520.

To determine a profile, shape, and or volume of a bubble region, one or more buccal-lingual planes 1530 may be projected into the region 1520. The example dental arch 1500 shows 4 buccal-lingual planes 1530, but in other examples, any number of buccal-lingual planes 1530 may be used. The buccal-lingual planes 1530 are generally normal to the dental arch 1500. Jaw arch splines may be projected from a patient's existing teeth through one or more of the buccal-lingual planes 1530. An example jaw arch spline 1510 is shown. In some examples, there may be five jaw arch splines corresponding to buccal gingiva, lingual gingiva, buccal cusp, lingual cusp, and groove jaw arch splines. Other examples may include more, fewer, and different jaw arch splines. The jaw arch splines may be piecewise defined polynomial equations that determined to pass through like points on each existing teeth.

Because the projected jaw arch splines are associated with prominent external features of the patient's existing teeth, the intersections of the jaw arch splines onto the buccal-lingual planes 1530 may reflect or predict possible external features of erupting teeth within the region 1520.

FIG. 15B shows a side view of the dental arch 1500. The buccal-lingual planes 1530 may be spaced within the region 1520. The jaw arch spline 1510 is shown intersecting the buccal-lingual planes 1530. As described with respect to FIG. 14, a number of jaw arch splines may be used to define a curve 1401 on the buccal-lingual planes 1530. The curve 1401 may be used to define a profile or shape of a void or bubble region of a dental aligner.

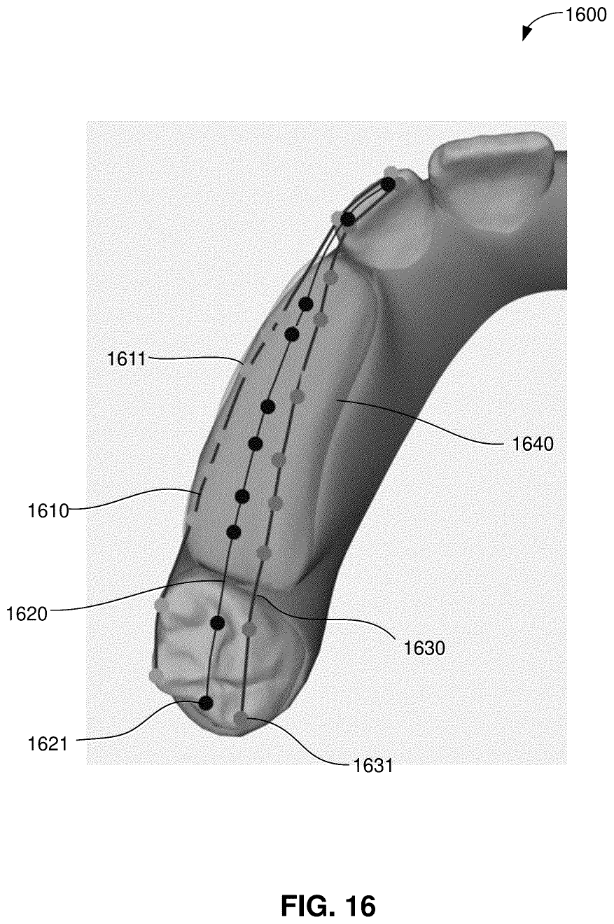

FIG. 16 shows a partial dental arch 1600 that may be displayed on a graphical user interface. The partial dental arch 1600 can include a first jaw arch spline 1610, a second jaw arch spline 1620, a third jaw arch spline 1630, and a bubble region 1640. In some cases, a clinician or other user may not be satisfied with the shape of the bubble region 1640. (Recall that the bubble region 1640 may be determined, at least in part, by jaw arch splines projected through a number of buccal-lingual planes.) In some implementations, a clinician or other user can adjust a position or trajectory of one or more jaw arch splines to modify the bubble region 1640. In some cases, a number of control points (depicted here as dots) associated with different jaw arch splines may be displayed on the graphical user interface. The control points may provide a “handle” for the clinician or user to interactively move, reposition, or adjust any jaw arch spline. Thus, by moving a control point via the graphical user interface, the clinician or user can modify a shape of the bubble region 1640. The bubble region 1640 may be updated on the graphical user interface as the clinician adjusts any jaw arch spline.

As shown, the first jaw spline 1610 can include one or more control points 1611, the second jaw spline 1620 can include one or more control points 1621, the third jaw spline 1630 can include one or more control points 1631. The control points 1611, 1621, and 1631 may be distributed on associated jar arch splines in any feasible manner. In some examples, the number of control points may be in proportion to a proposed size of the bubble region 1640. Thus, relatively larger bubble regions may have more control points and relatively smaller bubble regions may have fewer control points.

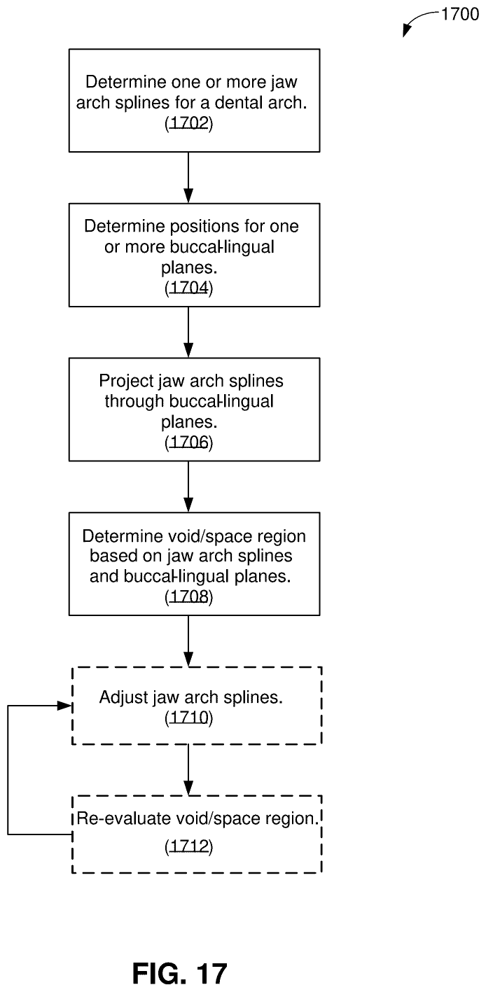

FIG. 17 is a flowchart showing one example of a method 1700 for determining a void in a dental aligner. The method 1700 is described below with respect to the apparatus 200 of FIG. 2 however, the method 1700 may be performed by any other suitable apparatus, system, or device.

The method begins in block 1702 as the processing node 210 determines one or more jaw arch splines for a dental arch. In some examples, dental arch information may be received through the API 250 or may be stored in the data storage module 240. The dental arch may be derived or determined from an x-ray image 220, or from any other data, including two-dimensional or three-dimensional dental scan data. Jaw arch splines may be determined to fit any particular dental arch. In some variations, the one or more jaw arch splines may be associated with particular aspects or characteristics of a tooth. Some aspects or characteristics may include buccal gingiva points, lingual gingiva points, buccal cusp points, lingual cusp points, and/or groove points or a tooth. The processing node 210 may determine a piecewise defined polynomial equation for each jaw arch spline.

Next, in block 1704 the processing node 210 determines positions for one or more buccal-lingual planes. The buccal-lingual planes may be positioned (virtually positioned) in areas of the dental arch that have or will have erupting teeth. The buccal-lingual planes are generally normal to the dental arch. The processing node 210 can place (define) any feasible number of buccal-lingual planes. In some cases, the buccal-lingual planes may be evenly spaced. In some other examples, the buccal-lingual planes may be unevenly spaced.

Next, in block 1706 the processing node 210 projects the jaw arch splines determined in block 1702 through the buccal-lingual planes determined in block 1704. Where the jaw arch splines intersect the buccal-lingual planes, a tooth profile (curve) may be defined.

Next, in block 1708 the processing node 210 determines a bubble region (e.g., void) for a dental aligner based on the jaw arch splines and the buccal-lingual planes. More particularly, the processing node 210 may determine a volume or shape for the void that is based on the tooth profiles (curves) that have been determined by the intersection of the jaw arch splines and the buccal-lingual planes. In some examples, the shape of the void may be determined by a geometric union of the various tooth profiles from each of the buccal-lingual planes.

Next, in block 1710 a clinician or other user can adjust one or more jaw arch splines. This operation is optional as denoted in FIG. 17 with dashed lines. In some examples, the processing node 210 may display on a graphical user interface the bubble region (void) as well as one or more of the jaw arch splines that are contributing to the shape of the bubble region. If the clinician would like to modify the shape of the bubble region, then the clinician can adjust one or more of the jaw arch splines. In some examples, the processing node 210 can display one or more control points on the jaw arch splines (through the graphical user interface) that the clinician can interact with to modify their trajectory.

Next, in block 1712, the clinician re-evaluates the bubble region. If the clinician is satisfied with the modified bubble region, the method 1700 ends. On the other hand, if the clinician is not satisfied with the modified bubble region, then the method returns to block 1710.

In some cases, the bubble region may interact or otherwise interfere with the patient's bite. For example, teeth from the opposite jaw (opposite dental arch) may touch or otherwise collide with the bubble region. This interaction may cause discomfort and aligner damage. In some variations, the shape of the teeth from the opposite jaw may be removed from the bubble region. The removal of the aligner material in the bubble region can reduce or eliminate unintentional interactions or collisions with the patient's teeth.

In general, these methods and apparatuses (systems, devices, etc., including software, hardware and/or firmware) for predicting tooth eruption, including any of the components described herein, as illustrated and described in FIGS. 2-8 and 10, discussed above, which may collectively be referred to as tooth eruption prediction modules, may be used at one or more parts of a dental computing environment, including as part of an intraoral scanning system, doctor system, treatment planning (e.g., technician) system, patient system, and/or fabrication system. In particular, these methods and apparatuses may be used as part of a treatment planning system that integrates dual-arch passive aligners into an orthodontic treatment plan.

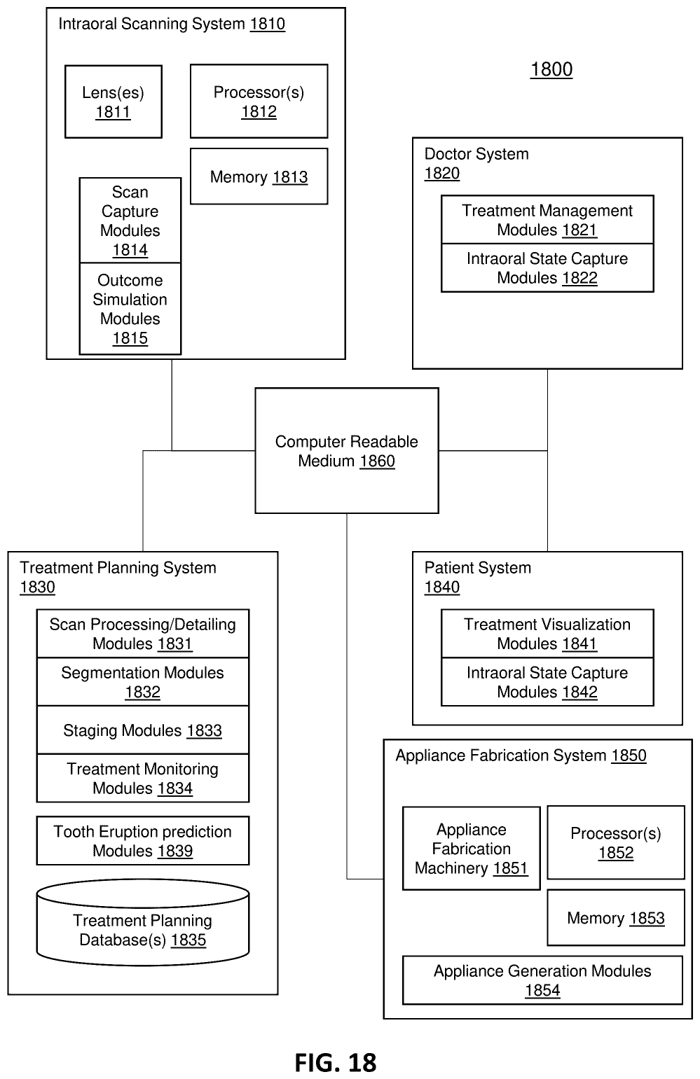

FIG. 18 is a diagram illustrating one variation of a computing environment 1800 that may generate one or more orthodontic/dental appliances and/or treatment plans specific to a patient, and fabricate dental appliances that may accomplish the treatment plan to treat a patient, under the direction of a dental professional. The example computing environment 1800 shown in FIG. 18 includes an intraoral scanning system 1810, a doctor system 1820, a treatment planning system 1830 (e.g., technician system), a patient system 1840, an appliance fabrication system 1850, and computer-readable medium 1860. Each of these systems may be referred to equivalently as a sub-system of the overall system (e.g., computing environment). Although shown as discrete systems, some or all of these systems may be integrated and/or combined. In some variations a computing environment (dental computing system) 1800 may include just one or a subset of these systems (which may also be referred to as sub-systems of the overall system 1800). As mentioned, one or more of these systems may be combined or integrated with one or more of the other systems (sub-systems), such as, e.g., the patient system and the doctor system may be part of a remote server accessible by doctor and/or patient interfaces. The computer readable medium 1860 may divided between all or some of the systems (subsystems); for example, the treatment planning system and appliance fabrication system may be part of the same sub-system and may be on a computer readable medium 1860. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, an intraoral scanning system 1810 can include optics 1811 (e.g., one or more lenses, filters, mirrors, etc.), processor(s) 1812, a memory 1813, scan capture module 1814, and outcome simulation module 1815. In general, the intraoral scanning system 1810 can capture one or more images of a patient's dentition. Use of the intraoral scanning system 1810 may be in a clinical setting (doctor's office or the like) or in a patient-selected setting (the patient's home, for example). In some cases, operations of the intraoral scanning system 1810 may be performed by an intraoral scanner, dental camera, cell phone or any other feasible device.

The optical components 1811 may include one or more lenses and optical sensors to capture reflected light, particularly from a patient's dentition. The scan capture module 1814 can include instructions (such as non-transitory computer-readable instructions) that may be stored in the memory 1813 and executed by the processor(s) 1812 to can control the capture of any number of images of the patient's dentition.

For example, the outcome simulation module 1815, which may be part of the intraoral scanning system 1810, can include instructions that simulate the tooth positions based on a treatment plan. Alternatively or additionally, in some examples, the outcome simulation module 1815 can import tooth number information from 3D models onto 2D images to assist in determining an outcome simulation.

Any of the component systems or sub-systems of the dental computing environment 1800 may access or use the 3D model of the patient's dentition. In addition, any of these sub-systems may use or access the tooth eruption prediction module(s) 1839 described above. For example, the doctor system 1820 may include a treatment management module 1821 and an intraoral state capture module 1822 that may access or use the 3D model and/or the tooth eruption prediction module(s) 1839. The doctor system 1820 may provide a “doctor facing” interface to the computing environment 1800. The treatment management module 1821 can perform any operations that enable a doctor or other clinician to manage the treatment of any patient. In some examples, the treatment management module 1821 may provide a visualization and/or simulation of the patient's dentition with respect to a treatment plan. This user interface may also include display and/or control of the eruption prediction as described above.

The intraoral state capture module 1822 can provide images of the patient's dentition to a clinician through the doctor system 1820. The images may be captured through the intraoral scanning system 1810 and may also include images of a simulation of tooth movement based on a treatment plan.

In some examples, the treatment management module 1821 can enable the doctor to modify or revise a treatment plan, particularly when images provided by the intraoral state capture module 1822 indicate that the movement of the patient's teeth may not be according to the treatment plan. The doctor system 1820 may include one or more processors configured to execute any feasible non-transitory computer-readable instructions to perform any feasible operations described herein.

Alternatively or additionally, the treatment planning system 1830 may include any of the methods and apparatuses described herein. The treatment planning system 1830 may include scan processing/detailing module 1831, segmentation module 1832, staging module 1833, treatment monitoring module 1834, tooth eruption prediction module 1839, and treatment planning database(s) 1835. In general, the treatment planning system 1830 can determine a treatment plan for any feasible patient. The scan processing/detailing module 1831 can receive or obtain dental scans (such as scans from the intraoral scanning system 1810) and can process the scans to “clean” them by removing scan errors and, in some cases, enhancing details of the scanned image. The treatment planning system 1830 may perform segmentation. For example, a treatment planning system may include a segmentation module 1832 that can segment a dental model into separate parts including separate teeth, gums, jaw bones, and the like. In some cases, the dental models may be based on scan data from the scan processing/detailing module 1831.

The staging module 1833 may determine different stages of a treatment plan. Each stage may correspond to a different dental aligner. The staging module 1833 may also determine the final position of the patient's teeth, in accordance with a treatment plan. Thus, the staging module 1833 can determine some or all of a patient's orthodontic treatment plan. In some examples, the staging module 1833 can simulate movement of a patient's teeth in accordance with the different stages of the patient's treatment plan. The staging module may also integrate with any of the tooth eruption prediction module(s) 1839 to account for tooth eruption and/or exfoliation.

The treatment monitoring module 1834 can monitor the progress of an orthodontic treatment plan. In some examples, the treatment monitoring module 1834 can provide an analysis of progress of treatment plans to a clinician.

Although not shown here, the treatment planning system 1830 can include one or more processors configured to execute any feasible non-transitory computer-readable instructions to perform any feasible operations described herein.

The patient system 1840 can include a treatment visualization module 1841 and an intraoral state capture module 1842. In general, the patient system 1840 can provide a “patient facing” interface to the computing environment 1800. The treatment visualization module 1841 can enable the patient to visualize how an orthodontic treatment plan has progressed and also visualize a predicted outcome (e.g., a final position of teeth).

In some examples, the patient system 1840 can capture dentition scans for the treatment visualization module 1841 through the intraoral state capture module 1842. The intraoral state capture module can enable a patient to capture his or her own dentition through the intraoral scanning system 1810. Although not shown here, the patient system 1840 can include one or more processors configured to execute any feasible non-transitory computer-readable instructions to perform any feasible operations described herein.

The appliance fabrication system 1850 can include appliance fabrication machinery 1851, processor(s) 1852, memory 1853, and appliance generation module 1854. In general, the appliance fabrication system 1850 can directly or indirectly fabricate aligners to implement an orthodontic treatment plan. In some examples, the orthodontic treatment plan may be stored in the treatment planning database(s) 1835.

The appliance fabrication machinery 1851 may include any feasible implement or apparatus that can fabricate any suitable dental aligner. The appliance generation module 1854 may include any non-transitory computer-readable instructions that, when executed by the processor(s) 1852, can direct the appliance fabrication machinery 1851 to produce one or more dental aligners. The memory 1853 may store data or instructions for use by the processor(s) 1852. In some examples, the memory 1853 may temporarily store a treatment plan, dental models, or intraoral scans.

The computer-readable medium 1860 may include some or all of the elements described herein with respect to the computing environment 1800. The computer-readable medium 1860 may include non-transitory computer-readable instructions that, when executed by a processor, can provide the functionality of any device, machine, or module described herein.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

A processor may include hardware that runs the computer program code. Specifically, the term ‘processor’ may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations, or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method, the method comprising:
extracting or receiving dental measurements from one or more x-ray images of a patient;
predicting a tooth eruption and/or exfoliation in the patient's dentition based on the dental measurements using a trained neural network, wherein the trained neural network is trained using a plurality of training x-ray images and corresponding patient data to generate a probability value indicating the likelihood of eruption and/or exfoliation of a tooth at a particular location; and
forming a dental appliance configured to fit over the patient's dentition and including an accommodation to accommodate the predicted tooth eruption and/or exfoliation, wherein the accommodation comprises one or more of: a cutout region configured to accommodate the predicted tooth eruption and/or exfoliation, a pocket configured to accommodate the predicted tooth eruption and/or exfoliation, and/or an undercut region configured to accommodate the predicted tooth eruption and/or exfoliation.

2. The method of claim 1, further comprising generating a treatment plan based on the probability value.

3. The method of claim 1, wherein the dental measurements include a crown to gingival distance for at least one tooth.

4. The method of claim 1, wherein the dental measurements include a relative distance between an unerupted tooth and a medial tooth's crown.

5. The method of claim 1, wherein the corresponding patient data includes patient age, patient gender, patient weight, patient ethnicity, patient geographic location, or a combination thereof.

6. The method of claim 1, wherein the corresponding patient data includes facial measurements associated with two or more facial datums.

7. The method of claim 1, wherein the x-ray images of the patient include panoramic x-rays, bitewing x-rays, or a combination thereof.

8. The method of claim 1, further comprising:
receiving updated dental measurements determined from one or more subsequent x-ray images of the patient; and
updating the prediction of the change in a patient's dentition based at least in part on the updated dental measurements.

9. The method of claim 8, wherein the updated dental measurements include measurements determined from an intraoral scan.

10. The method of claim 1, further comprising outputting the probability value for the particular location.

11. The method of claim 1, wherein forming the dental appliance comprises forming the dental appliance by a direct fabrication process.

12. The method of claim 1, wherein the dental appliance comprises a patient-removable aligner configured to fit over the patient's dentition.

13. The method of claim 1, wherein forming the dental appliance includes generating a digital model of the dental appliance and positioning the accommodation on the dental appliance in a region configured to be worn over the particular location.

14. An apparatus, the apparatus comprising:
one or more processors; and
a memory storing instructions that, when executed by the one or more processors, causes the apparatus to perform the method comprising:
extracting or receiving dental measurements from one or more x-ray images of a patient;
predicting a tooth eruption and/or exfoliation in the patient's dentition based on the dental measurements using a trained neural network, wherein the trained neural network is trained using a plurality of training x-ray images and corresponding patient data to generate a probability value indicating the likelihood of eruption and/or exfoliation of a tooth at a particular location;
forming a dental appliance configured to accommodate the predicted tooth eruption and/or exfoliation, wherein the dental appliance comprises one or more of: a cutout region configured to accommodate the predicted tooth eruption and/or exfoliation, a pocket configured to accommodate the predicted tooth eruption and/or exfoliation, and/or an undercut region configured to accommodate the predicted tooth eruption and/or exfoliation.

15. A method comprising:
positioning one or more jaw arch splines relative to a patient's dental arch in a virtual model of the patient's dental arch;
positioning one or more buccal-lingual planes in a region of the patient's dental arch to receive erupting teeth;
determining, for each buccal-lingual plane, a two-dimensional (2D) tooth profile based on a projection of the one or more jaw arch splines through the one or more buccal-lingual planes; and
forming a dental aligner configured to be worn over the patient's dental arch, wherein forming includes setting a void region for the dental aligner based on the 2D tooth profiles from the one or more buccal-lingual planes.

16. The method of claim 15, wherein the one or more jaw arch splines pass through an exterior point on a surface of existing teeth of the patient.

17. The method of claim 15, wherein the one or more jaw arch splines are associated with at least one of a buccal gingiva point or a lingual gingiva point on a surface of an existing tooth of the patient.

18. The method of claim 15, wherein the one or more jaw arch splines are associated with at least one of a buccal cusp, a lingual cusp, or a groove arch points on a surface of an existing tooth of the patient.

19. The method of claim 15, wherein the one or more jaw arch splines are described by piecewise continuous polynomial that describes a curve passing through a predetermined point of an existing tooth of the patient.

20. The method of claim 15, wherein the one or more buccal-lingual planes are normal to a patient's dental arch.

21. The method of claim 15, wherein determining the void includes forming a volume based on the projection of the one or more jaw arch splines through the one or more buccal-lingual planes.

22. The method of claim 15, wherein determining the one or more jaw arch splines comprises adjusting, by a clinician, the one or more jaw arch splines.

23. The method of claim 22, wherein the adjusting is performed by the clinician interacting with a graphical user interface.

24. The method of claim 22, further comprising displaying the one or more jaw arch splines on a graphical user interface.

25. The method of claim 15, further comprising displaying the dental aligner including the void on a display.

26. The method of claim 15, further comprising modifying the dental aligner to accommodate teeth from a different dental arch.

27. The method of claim 15, wherein forming the dental appliance comprises forming the dental appliance by a direct fabrication process.
